(19) United States
(12) Patent Application Publication
(10) Pub. No.: US 2011/0105857 A1
(43) Pub. Date: May 5, 2011

(54) IMPRESSION DEGREE EXTRACTION APPARATUS AND IMPRESSION DEGREE EXTRACTION METHOD

(75) Inventors: Wenli Zhang, Kanagawa (JP); Koichi Emura, Kanagawa (JP); Sachiko Uranaka, Tokyo (JP)

(73) Assignee: PANASONIC CORPORATION, Osaka (JP)

(21) Appl. No.: 13/001,459

(22) PCT Filed: Apr. 14, 2009
(86) PCT No.: PCT/JP2009/001723

§ 371 Date: Dec. 27, 2010

(30) Foreign Application Priority Data
Jul. 3, 2008 (JP) 2008-174763

(51) Int. Cl. A61B 5/00

(52) U.S. Cl. 600/300

(57) ABSTRACT

An impression degree extraction apparatus which precisely extracts an impression degree without imposing a strain on a user in particular. A content editing apparatus (100) comprises a measured emotion property acquiring section (341) which acquires measured emotion properties which show an emotion having occurred in the user in a measurement period, and an impression degree calculating part (340) which calculates the impression degree being a degree which shows how strong the user was impressed in the measurement period by comparing reference emotion properties which shows an emotion having occurred in the user in a reference period and the measured emotion properties. The impression degree calculating part (340) calculates the impression degree to be higher with the increase of the difference between the first emotion properties and the second emotion properties with the second emotion properties as the reference.

### TECHNICAL FIELD

The present invention relates to an impression degree extraction apparatus and impression degree extraction method that extract an impression degree that is a degree indicating the intensity of an impression received by a user.

### BACKGROUND ART

When selecting images to be kept from among a large number of photographic images or when performing a selective operation in a game, for example, selection is often performed based on the intensity of an impression received by a user. However, when the number of objects is large, the selection process is burdensome for a user.

For example, with wearable type video cameras that have attracted attention in recent years, it is easy to perform continuous shooting over a long period, such as throughout an entire day. However, when such lengthy shooting is performed, a major problem is how to pick out parts that are important to a user from a large amount of recorded video data. A part that is important to a user should be decided based on the subjective feelings of the user. Therefore, it is necessary to carry out tasks of searching and summarization of important parts while checking video in its entirety.

Thus, a technology that automatically selects video based on a user's arousal level has been described in Patent Literature 1, for example. With the technology described in Patent Literature 1, a user's brainwaves are recorded in synchronization with video shooting, and automatic video editing is performed by extracting sections of shot video for which the user's arousal level is higher than a predetermined reference value. By this means, video selection can be automated, and the burden on a user can be alleviated.

### CITATION LIST

### Patent Literature

PTL 1
Japanese Patent Application Laid-Open No.2002-204419

### SUMMARY OF INVENTION

### Technical Problem

However, with a comparison between an arousal level and a reference value, only degrees of excitement, attention, and concentration can be determined, and it is difficult to determine the higher-level emotional states of delight, anger, sorrow, and pleasure. Also, there are individual differences in an arousal level that is a criterion for selection. Furthermore, the intensity of an impression received by a user may appear as the way in which an arousal level changes rather than an arousal level itself. Therefore, with the technology described in Patent Literature 1, a degree indicating the intensity of an impression received by a user (hereinafter referred to as “impression degree”) cannot be extracted with a high degree of precision, and there is a high probability of not being able to obtain selection results that satisfy a user. For example, with the above-described automatic editing of shot video, it is difficult to accurately extract scenes that leave an impression. In this case, it may be necessary for the user to redo the selection process manually while checking the selection results, thereby imposing a burden on the user.

It is an object of the present invention to provide an impression degree extraction apparatus and impression degree extraction method that enable an impression degree to be extracted with a high degree of precision without particularly imposing a burden on a user.

### Solution to Problem

An impression degree extraction apparatus of the present invention has a first emotion characteristic acquisition section that acquires a first emotion characteristic indicating a characteristic of an emotion that has occurred in a user in a first period, and an impression degree calculation section that calculates an impression degree that is a degree indicating the intensity of an impression received by the user in the first period by means of a comparison of a second emotion characteristic indicating a characteristic of an emotion that has occurred in the user in a second period different from the first period with the first emotion characteristic.

An impression degree extraction method of the present invention has a step of acquiring a first emotion characteristic indicating a characteristic of an emotion that has occurred in a user in a first period, and a step of calculating an impression degree that is a degree indicating the intensity of an impression received by the user in the first period by means of a comparison of a second emotion characteristic indicating a characteristic of an emotion that has occurred in the user in a second period different from the first period with the first emotion characteristic.

### Advantageous Effects of Invention

The present invention enables an impression degree of a first period to be calculated taking the intensity of an impression actually received by a user in a second period as a comparative criterion, thereby enabling an impression degree to be extracted with a high degree of precision without particularly imposing a burden on the user.

### BRIEF DESCRIPTION OF DRAWINGS

FIG. 1 is a block diagram of a content editing apparatus that includes an impression degree extraction apparatus according to Embodiment 1 of the present invention;

FIG. 2 is a drawing showing an example of a two-dimensional emotion model used in a content editing apparatus according to Embodiment 1;

FIG. 3 is a drawing for explaining an emotion measured value in Embodiment 1;

FIG. 4 is a drawing showing the nature of time variation of an emotion in Embodiment 1;

FIG. 5 is a drawing for explaining an emotion amount in Embodiment 1;

FIG. 6 is a drawing for explaining an emotion transition direction in Embodiment 1;

FIG. 7 is a drawing for explaining emotion transition velocity in Embodiment 1;

FIG. 8 is a sequence diagram showing an example of the overall operation of a content editing apparatus according to Embodiment 1;

FIG. 9 is a flowchart showing an example of emotion information acquisition processing in Embodiment 1;

FIG. 10 is a drawing showing an example of emotion information history contents in Embodiment 1;

FIG. 11 is a flowchart showing reference emotion characteristic acquisition processing in Embodiment 1;

FIG. 12 is a flowchart showing emotion transition information acquisition processing in Embodiment 1;

FIG. 13 is a drawing showing an example of reference emotion characteristic contents in Embodiment 1;

FIG. 14 is a drawing showing an example of emotion information data contents in Embodiment 1;

FIG. 15 is a flowchart showing impression degree calculation processing in Embodiment 1;

FIG. 16 is a flowchart showing an example of difference calculation processing in Embodiment 1;

FIG. 17 is a drawing showing an example of impression degree information contents in Embodiment 1;

FIG. 18 is a flowchart showing an example of experience video editing processing in Embodiment 1;

FIG. 19 is a block diagram of a game terminal that includes an impression degree extraction apparatus according to Embodiment 2 of the present invention;

FIG. 20 is a flowchart showing an example of content manipulation processing in Embodiment 2;

FIG. 21 is a block diagram of a mobile phone that includes an impression degree extraction apparatus according to Embodiment 3 of the present invention;

FIG. 22 is a flowchart showing an example of screen design change processing in Embodiment 3;

FIG. 23 is a block diagram of a communication system that includes an impression degree extraction apparatus according to Embodiment 4 of the present invention;

FIG. 24 is a flowchart showing an example of accessory change processing in Embodiment 4;

FIG. 25 is a block diagram of a content editing apparatus that includes an impression degree extraction apparatus according to Embodiment 5 of the present invention;

FIG. 26 is a drawing showing an example of a user input screen in Embodiment 5; and

FIG. 27 is a drawing for explaining an effect in Embodiment 5.

### DESCRIPTION OF EMBODIMENTS

Now, embodiments of the present invention will be described in detail with reference to the accompanying drawings.

### Embodiment 1

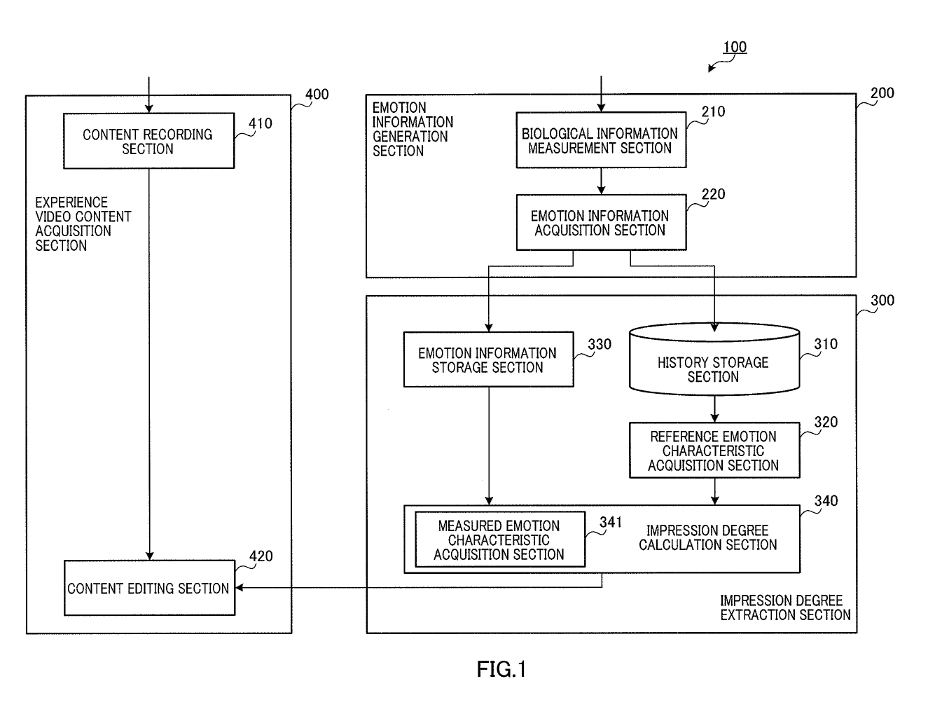

FIG. 1 is a block diagram of a content editing apparatus that includes an impression degree extraction apparatus according to Embodiment 1 of the present invention. This embodiment of the present invention is an example of application to an apparatus that performs video shooting using a wearable video camera at an amusement park or on a trip, and edits the shot video (hereinafter referred to for convenience as “experience video content”).

In FIG. 1, content editing apparatus 100 broadly comprises emotion information generation section 200, impression degree extraction section 300, and experience video content acquisition section 400.

Emotion information generation section 200 generates emotion information indicating an emotion that has occurred in a user from the user's biological information. Here, “emotion” denotes not only an emotion of delight, anger, sorrow, or pleasure, but also a general psychological state, including a feeling such as relaxation. Emotion information is an object of impression degree extraction by impression degree extraction section 300, and will be described in detail later herein. Emotion information generation section 200 has biological information measurement section 210 and emotion information acquisition section 220.

Biological information measurement section 210 is connected to a detection apparatus such as a sensor, digital camera, or the like (not shown), and measures a user's biological information. Biological information includes, for example, at least one of the following: heart rate, pulse, body temperature, facial myoelectrical signal, and voice.

Emotion information acquisition section 220 generates emotion information from a user's biological information obtained by biological information measurement section 210.

Impression degree extraction section 300 extracts an impression degree based on emotion information generated by emotion information acquisition section 220. Here, an impression degree is a degree indicating the intensity of an impression received by a user in an arbitrary period when the intensity of an impression received by the user in a past period that is a reference for the user's emotion information (hereinafter referred to as “reference period”) is taken as a reference. That is to say, an impression degree is the relative intensity of an impression when the intensity of an impression in a reference period is taken as a reference. Therefore, by making a reference time a period in which a user is in a normal state, or a sufficiently long period, an impression degree becomes a value that indicates a degree of specialness different from a normal state. In this embodiment, a period in which experience video content is recorded is assumed to be a period that is an object of impression degree extraction (hereinafter referred to as “measurement period”). Impression degree extraction section 300 has history storage section 310, reference emotion characteristic acquisition section 320, emotion information storage section 330, and impression degree calculation section 340.

History storage section 310 accumulates emotion information acquired in the past by emotion information generation section 200 as an emotion information history.

Reference emotion characteristic acquisition section 320 reads emotion information of a reference period from the emotion information history stored in history storage section 310, and generates information indicating a characteristic of a user's emotion information in the reference period (hereinafter referred to as a “reference emotion characteristic”) from the read emotion information.

Emotion information storage section 330 stores emotion information obtained by emotion information generation section 200 in a measurement period.

Impression degree calculation section 340 calculates an impression degree based on a difference between information indicating a characteristic of user's emotion information in the measurement period (hereinafter referred to as a “measured emotion characteristic”) and a reference emotion characteristic calculated by reference emotion characteristic acquisition section 320. Impression degree calculation section 340 has measured emotion characteristic acquisition section 341 that generates a measured emotion characteristic from emotion information stored in emotion information storage section 330.

Experience video content acquisition section 400 records experience video content, and performs experience video content editing based on an impression degree calculated from emotion information during recording (in the measurement period). Experience video content acquisition section 400 has content recording section 410 and content editing section 420. The impression degree will be described later in detail.

Content recording section 410 is connected to a video input apparatus such as a digital video camera (not shown), and records experience video shot by the video input apparatus as experience video content.

Content editing section 420, for example, compares an impression degree obtained by impression degree extraction section 300 with experience video content recorded by content recording section 410 by mutually associating them on the time axis, extracts a scene corresponding to a period in which an impression degree is high, and generates a summary video of experience video content.

Content editing apparatus 100 has, for example, a CPU (central processing unit), a storage medium such as ROM (read only memory) that stores a control program, working memory such as RAM (random access memory), and so forth. In this case, the functions of the above sections are implemented by execution of the control program by the CPU.

According to content editing apparatus 100 of this kind, an impression degree is calculated by means of a comparison of characteristic values based on biological information, and therefore an impression degree can be extracted without particularly imposing a burden on a user. Also, an impression degree is calculated taking a reference emotion characteristic obtained from biological information of a user himself in a reference period as a reference, enabling an impression degree to be calculated with a high degree of precision. Furthermore, a summary video is generated by selecting a scene from experience video content based on an impression degree, enabling experience video content to be edited by picking up only a scene with which a user is satisfied. Moreover, since an impression degree is extracted with a high degree of precision, content editing results with which a user is more satisfied can be obtained, and the necessity of a user performing re-editing can be reduced.

Before giving a description of the operation of content editing apparatus 100, the various kinds of information used by content editing apparatus 100 will now be described.

First, an emotion model used when defining emotion information quantitatively will be described.

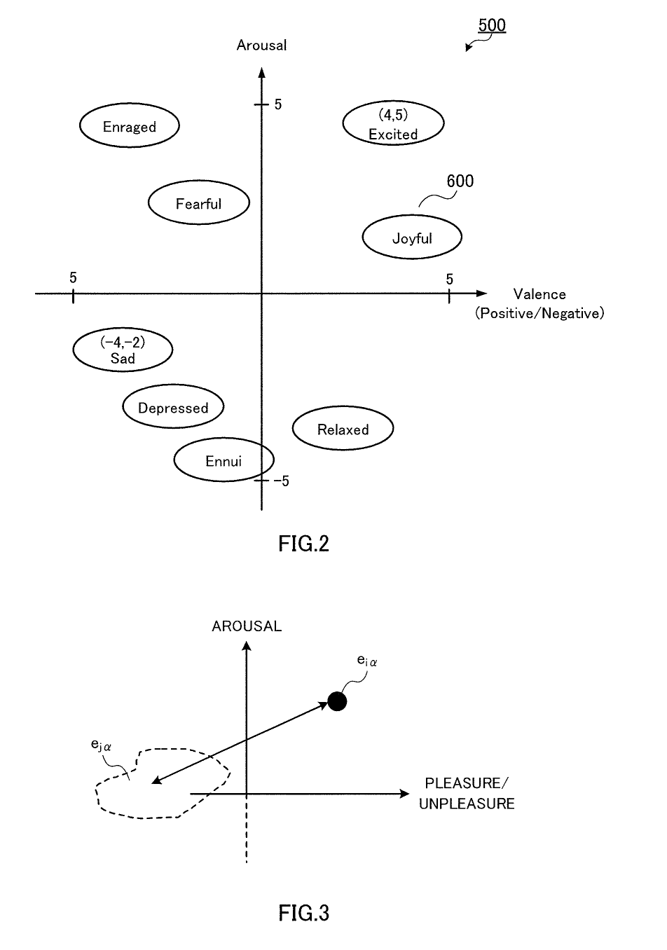

FIG. 2 is a drawing showing an example of a two-dimensional emotion model used in content editing apparatus 100.

Two-dimensional emotion model 500 shown in FIG. 2 is an emotion model called a LANG emotion model. Two-dimensional emotion model 500 comprises two axes: a horizontal axis indicating valence, which is a degree of pleasure or unpleasure (or positive emotion or negative emotion), and a vertical access indicating arousal, which is a degree of excitement/tension or relaxation. In the two-dimensional space of two-dimensional emotion model 500, regions are defined by emotion type, such as “Excited”, “Relaxed”, “Sad”, and so forth, according to the relationship between the horizontal and vertical axes. Using two-dimensional emotion model 500, an emotion can easily be represented by a combination of a horizontal axis value and vertical axis value. Emotion information in this embodiment comprises coordinate values in this two-dimensional emotion model 500, indirectly representing an emotion.

Here, for example, coordinate values (4,5) denote a position in a region of the emotion type “Excited”, and Also, coordinate values (−4,−2) denote a position in a region of the emotion type “Sad”.

Therefore, an emotion expected value and emotion measured value comprising coordinate values (4,5) indicate the emotion type “Excited”, and an emotion expected value and emotion measured value comprising coordinate values (−4,−2) indicate the emotion type “Sad”. When the distance between an emotion expected value and emotion measured value in two-dimensional emotion model 500 is short, the emotions indicated by each can be said to be similar. Emotion information of this embodiment is assumed to be information in which a time at which biological information that is the basis of an emotion measured value has been added to that emotion measured value.

A model with more than two dimensions or a model other than a LANG emotion model may also be used as an emotion model. For example, content editing apparatus 100 may use a three-dimensional emotion model (pleasure/unpleasure, excitement/calmness, tension/relaxation) or a six-dimensional emotion model (anger, fear, sadness, delight, dislike, surprise) as an emotion model. Using such an emotion model with more dimensions enables emotion types to be represented more precisely.

Next, types of parameters composing a reference emotion characteristic and measured emotion characteristic will be described using FIG. 3 through FIG. 7. Parameter types composing a reference emotion characteristic and a measured emotion characteristic are the same, and include an emotion measured value, emotion amount, and emotion transition information. Emotion transition information includes emotion transition direction and emotion transition velocity. Below, symbol “e” indicates a parameter relating to a measured emotion characteristic; symbol “i” is a symbol indicating a parameter relating to a measured emotion characteristic, and is also a variable for identifying an individual measured emotion characteristic; and symbol “j” is a symbol indicating a parameter relating to a reference emotion characteristic, and is also a variable for identifying an individual reference emotion characteristic.

FIG. 3 is a drawing for explaining an emotion measured value. Emotion measured values e_{1α} and e_{jα} are coordinate values in two-dimensional emotion model 500 shown in FIG. 2, are expressed by (x,y). As shown in FIG. 3, if the coordinates of reference emotion characteristic emotion measured value e_{jα} are designated (xⱼ, yⱼ), and the coordinates of measured emotion characteristic emotion measured value e_{iα} are designated (xᵢ, yᵢ), emotion measured value difference r_{α} between the reference emotion characteristic and measured emotion characteristic is a value given by equation 1 below.

[1]

r_{α}=\sqrt{(xᵢ−xⱼ)²+(yᵢ−yⱼ)²} (Equation 1)

That is to say, emotion measured value difference r_{α} indicates a distance in the emotion model space—that is, the magnitude of a difference of emotion.

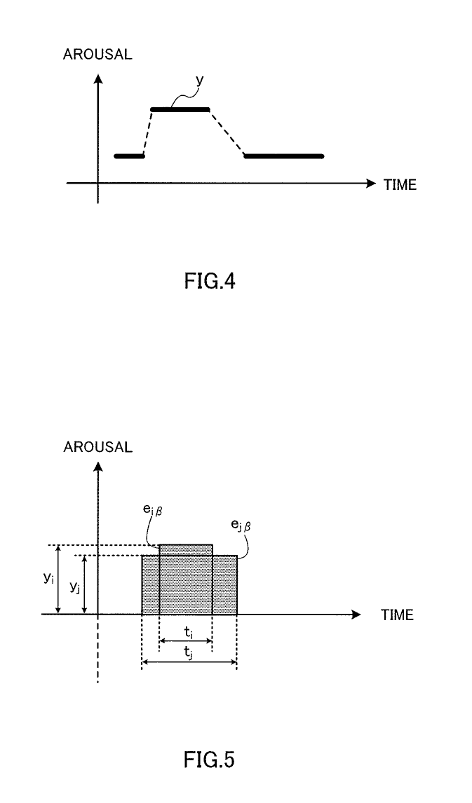

FIG. 4 is a drawing showing the nature of time variation of an emotion. Here, arousal value y (hereinafter referred to as “emotion intensity” for convenience) will be focused upon among emotion measured values as one characteristic indicating an emotional state. As shown in FIG. 4, emotion intensity y changes with the passage of time. Emotion intensity y becomes a high value when a user is excited or tense, and becomes a low value when a user is relaxed. Also, when a user continues to be excited or tense for a long time, emotion intensity y remains high for a long time. Even with the same emotion intensity, continuation for a long time can be said to indicate a more intense state of excitement. Therefore, in this embodiment, an emotion amount obtained by time integration of emotion intensity is used for impression value calculation.

FIG. 5 is a drawing for explaining an emotion amount. Emotion amounts e_{iβ} and e_{jβ} are values obtained by time integration of emotion intensity y. If the same emotion intensity y continues for time t, for example, emotion amount e_{iβ} is expressed by y×t. In FIG. 5, if a reference emotion characteristic emotion amount is designated yⱼ×tⱼ, and a measured emotion characteristic emotion amount is designated yᵢ×tᵢ, emotion amount difference r_{β} between the reference emotion characteristic and measured emotion characteristic is a value given by equation 2 below.

[2]

r_{β}=(yᵢ×tᵢ)−(yⱼ×tⱼ) (Equation 2)

That is to say, emotion amount difference r_{β} indicates a difference in emotion intensity integral values—that is, a difference in emotion intensity.

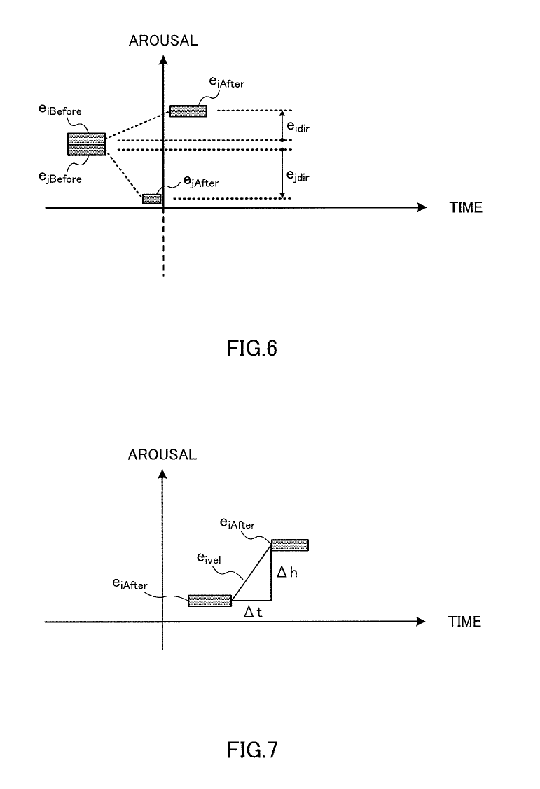

FIG. 6 is a drawing for explaining an emotion transition direction. Emotion transition directions e_{idir} and e_{jdir} are information indicating a transition direction when an emotion measured value makes a transition using a pair of emotion measured values before and after the transition. Here, a pair of emotion measured values before and after the transition is, for example, a pair of emotion measured values acquired at a predetermined time interval, and is here assumed to be a pair of emotion measured values obtained successively. In FIG. 6, only arousal (emotion intensity) is focused upon, and emotion transition directions e_{idir} and e_{jdir} are shown. If, for example, an emotion measured value that is an object of processing is designated e_{iAfter}, and the immediately preceding emotion measured value is designated e_{iBefore}, emotion transition direction e_{idir} is a value given by equation 3 below.

[3]

e_{idir}=e_{iAfter}−e_{iBefore} (Equation 3)

Emotion transition direction e_{jdir} can be found in a similar way from emotion measured values e_{jAfter} and e_{jBefore}.

FIG. 7 is a drawing for explaining emotion transition velocity. Emotion transition velocities eᵢᵥₑₗ and eⱼᵥₑₗ are information indicating transition velocity when an emotion measured value makes a transition using a pair of emotion measured values before and after the transition. In FIG. 7, only arousal (emotion intensity) is focused upon, and only parameters relating to a measured emotion characteristic are focused upon and shown. If, for example, a transition width of emotion intensity is designated Δh, and a time necessary for transition is designated Δt (an emotion measured value acquisition interval), emotion transition velocity eᵢᵥₑₗ is a value given by equation 4 below.

[4]

eᵢᵥₑₗ=|e_{iAfter}−e_{iBefore}|/Δt=Δh/Δt (Equation 4)

Emotion transition direction eⱼᵥₑₗ can be found in a similar way from emotion measured values e_{jAfter} and e_{jBefore}.

Emotion transition information is a value obtained by weighting and adding an emotion transition direction and emotion transition velocity. When a weight of emotion transition direction e_{idir} is designated w_{idir}, and a weight of emotion transition velocity eᵢᵥₑₗ is designated wᵢᵥₑₗ, emotion transition information e_{iδ} is a value given by equation 5 below.

[5]

e_{iδ}=e_{idir}×w_{idir}+eᵢᵥₑₗ×wᵢᵥₑₗ (Equation 5)

Emotion transition information e_{jδ} can be found in a similar way from weight of emotion transition direction e_{jdir} and its weight w_{idir}, and weight of emotion transition velocity eⱼᵥₑₗ and its weight wⱼᵥₑₗ.

Emotion transition information difference r_{δ} between a reference emotion characteristic and measured emotion characteristic is a value given by equation 6 below.

[6]

r_{δ}=e_{iδ}−e_{jε} (Equation 6)

That is to say, emotion transition information difference r_{δ} indicates a degree of difference according to the nature of an emotion transition.

Calculating such an emotion measured value difference r_{α}, emotion amount difference r_{β}, and emotion transition information difference r_{δ}, enables a difference in emotion between a reference period and a measurement period to be determined with a high degree of precision. For example, it is possible to detect psychological states characteristic of receiving a strong impression, such as the highly emotional states of delight, anger, sorrow, and pleasure, the duration of a state in which emotion is heightened, a state in which a usually calm person suddenly becomes excited, a transition from a “sad” state to a “joyful” state, and so forth.

Next, the overall operation of content editing apparatus 100 will be described.

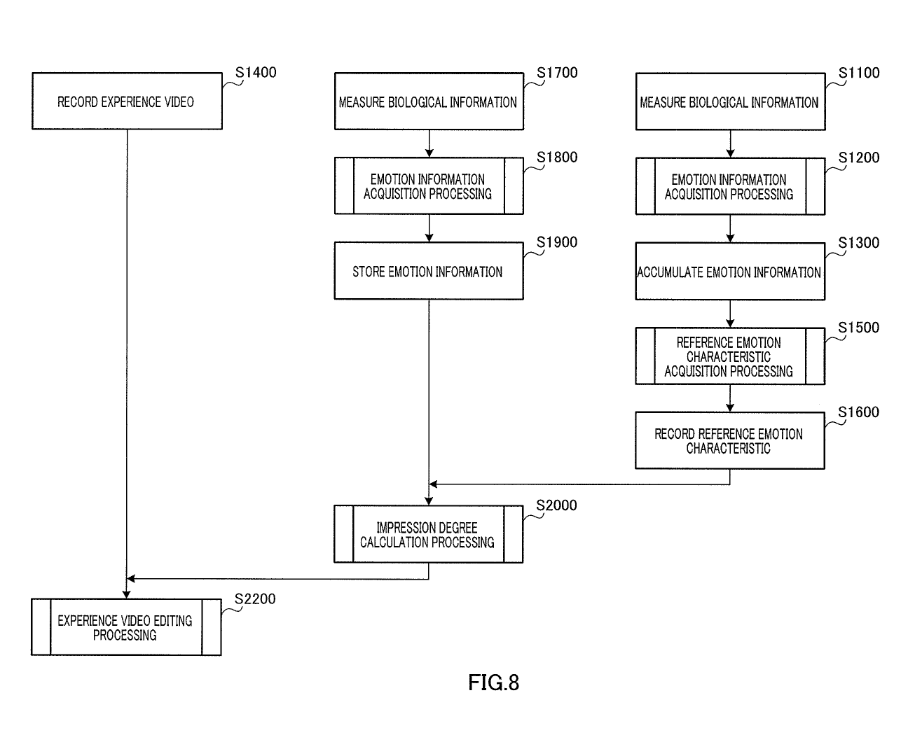

FIG. 8 is a sequence diagram showing an example of the overall operation of content editing apparatus 100.

The operation of content editing apparatus 100 broadly comprises two stages: a stage in which emotion information that is the basis of a reference emotion characteristic is accumulated (hereinafter referred to as an “emotion information accumulation stage”), and a stage in which content is edited based on emotion information measured in real time (hereinafter referred to as a “content editing stage”). In FIG. 8, steps S1100 through S1300 are emotion information accumulation stage processing, and steps S1400 through S2200 are content editing stage processing.

First, emotion information accumulation stage processing will be described.

Prior to processing, a sensor for detection of necessary biological information from a user and a digital video camera for shooting video are set. When setting is completed, operation of content editing apparatus 100 is started.

First, in step S1100, biological information measurement section 210 measures a user's biological information, and outputs the acquired biological information to emotion information acquisition section 220. As biological information, biological information measurement section 210 detects, for example, at least one of the following: brainwaves, electrical skin resistance, skin conductance, skin temperature, electrocardiographic frequency, heart rate, pulse, body temperature, a myoelectrical signal, a facial image, voice, and so forth.

Then, in step S1200, emotion information acquisition section 220 starts emotion information acquisition processing. Emotion information acquisition processing is processing whereby, at predetermined intervals, biological information is analyzed, and emotion information is generated and output to impression degree extraction section 300.

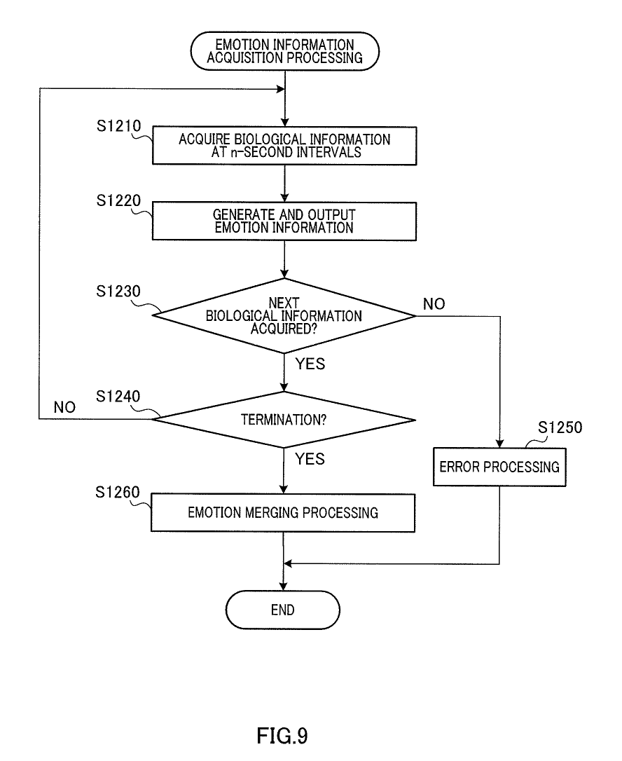

FIG. 9 is a flowchart showing an example of emotion information acquisition processing.

First, in step S1210, emotion information acquisition section 220 acquires biological information from biological information measurement section 210 at a predetermined time interval (assumed here to be an interval of n seconds).

Then, in step S1220, emotion information acquisition section 220 acquires an emotion measured value based on biological information, generates emotion information from the emotion measured value, and outputs this emotion information to impression degree extraction section 300.

The actual method of acquiring an emotion measured value from biological information, and contents represented by an emotion measured value, will now be described.

A biosignal of a person is known to change according to a change in a person's emotion. Emotion information acquisition section 220 acquires an emotion measured value from biological information using this relationship between a change in emotion and biosignal change.

For example, it is known that the more relaxed a person is, the greater is the proportion of an alpha (α) wave component. It is also known that an electrical skin resistance value is increased by surprise, fear, or anxiety, that skin temperature and electrocardiographic frequency are increased by a major occurrence of the emotion of joy, that heart rate and pulse show slow changes when a person is psychologically and emotionally stable, and so forth. It is further known that, apart from the above biological indicators, a type of expression and voice change in terms of crying, laughing, being angry, and so forth, according to emotions such as delight, anger, sorrow, and pleasure. Moreover, it is known that a person's voice tends to become quieter when that person is depressed, and to become louder when that person is angry or joyful.

Therefore, it is possible to detect an electrical skin resistance value, skin temperature, electrocardiographic frequency, heart rate, pulse, and voice level, analyze the proportion of an alpha wave component of brainwaves from brainwaves, perform expression recognition from a facial myoelectrical signal or facial image, perform voice recognition, and so forth, and acquire biological information, and to analyze an emotion from the biological information.

Specifically, for example, a conversion table or conversion equation for converting the above biological information values to coordinate values of two-dimensional emotion model 500 shown in FIG. 2 is prepared beforehand in emotion information acquisition section 220. Then emotion information acquisition section 220 maps emotion information input from biological information measurement section 210 onto the two-dimensional space of two-dimensional emotion model 500 using the conversion table or conversion equation, and acquires the relevant coordinate values as emotion measured values.

For example, skin conductance increases according to arousal, and electromyography (EMG) changes according to pleasure. Therefore, emotion information acquisition section 220 establishes correspondence to a degree of desirability for a user's experience contents (date, trip, or the like) at the time of experience video shooting, and measures skin conductance beforehand. By this means, correspondence can be established in two-dimensional emotion model 500 on a vertical axis indicating a skin conductance value as arousal and a horizontal axis indicating an electromyography value as pleasure. By preparing these correspondences beforehand as a conversion table or conversion equation, and detecting skin conductance and electromyography, an emotion measured value can easily be acquired.

An actual method of mapping biological information onto an emotion model space is described in “Emotion Recognition from Electromyography and Skin Conductance” (Arturo Nakasone, Helmut Prendinger, Mitsuru Ishizuka, The Fifth International Workshop on Biosignal Interpretation, BSI-05, Tokyo, Japan, 2005, pp. 219-222).

In this mapping method, correspondence to arousal and pleasure is first established using skin conductance and electromyography as biosignals. Mapping is performed based on the result of this correspondence using a probability model (Bayesian network) and 2-dimensional Lang emotion space model, and user emotion estimation is performed by means of this mapping. More specifically, skin conductance that increases linearly according to a person's degree of arousal, and electromyography that is related to pleasure (valence) indicating muscular activity, are measured when the user is in a normal state, the measurement results are taken as baseline values. That is to say, a baseline value represents biological information for a normal state. Next, when a user's emotion is measured, an arousal value is decided based on the degree to which skin conductance exceeds the baseline value. For example, if skin conductance exceeds the baseline value by 15% to 30%, arousal is determined to be very high. On the other hand, a valence value is decided based on the degree to which electromyography exceeds the baseline value. For example, if electromyography exceeds the baseline value by 3 times or more, valence is determined to be high, and if electromyography exceeds the baseline value by not more than 3 times, valence is determined to be normal. Then mapping of the calculated arousal value and valence value is performed using a probability model and 2-dimensional Lang emotion space model, and user emotion estimation is performed.

In step S1230 in FIG. 9, emotion information acquisition section 220 determines whether or not biological information after the next n seconds has been acquired by biological information measurement section 210. If the next biological information has been acquired (step S1230: YES), emotion information acquisition section 220 proceeds to step S1240, whereas if the next biological information has not been acquired (step S1230: NO), emotion information acquisition section 220 proceeds to step S1250.

In step S1250, emotion information acquisition section 220 executes predetermined processing such as notifying the user that an error has occurred in biological information acquisition, and terminates the series of processing steps.

On the other hand, in step S1240, emotion information acquisition section 220 determines whether or not termination of emotion information acquisition processing has been directed, and returns to step S1210 if termination has not been directed (step S1230: NO), or proceeds to step S1260 if termination has been directed (step S1240: YES).

In step S1260, emotion information acquisition section 220 executes emotion merging processing, and then terminates the series of processing steps. Emotion merging processing is processing whereby, when the same emotion measured value has been measured consecutively, those emotion measured values are merged into one item of emotion information. Emotion merging processing need not necessarily be performed.

By means of this kind of emotion information acquisition processing, emotion information is input to impression degree extraction section 300 each time an emotion measured value changes when merging processing is performed, or every n seconds when merging processing is not performed.

In step S1300 in FIG. 8, history storage section 310 accumulates input emotion information, and generates an emotion information history.

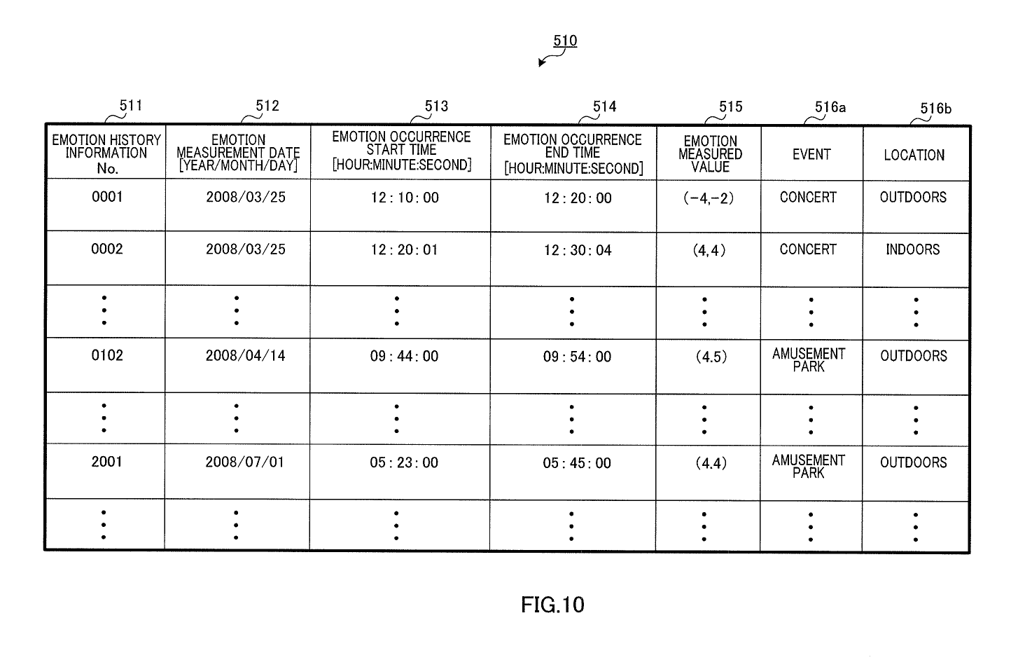

FIG. 10 is a drawing showing an example of emotion information history contents.

As shown in FIG. 10, history storage section 310 generates emotion information history 510 comprising records in which other information has been added to input emotion information. Emotion information history 510 includes Emotion History Information Number (No.) 511, Emotion Measurement Date [Year/Month/Day] 512, Emotion Occurrence Start Time [Hour:Minute:Second] 513, Emotion Occurrence End Time [Hour:Minute:Second] 514, Emotion Measured Value 515, Event 516a, and Location 516b.

A day on which measurement is performed is written in Emotion Measurement Date 512. If, for example, “2008/03/25” to “2008/07/01” are written in emotion information history 510 as Emotion Measurement Date 512, this indicates that emotion information acquired in this period (here, approximately three months) has been accumulated.

If the same emotion measured value (an emotion measured value written in Emotion Measured Value 515) has been measured consecutively, the start time of that measurement time—that is, the time in which an emotion indicated by that emotion measured value occurred—is written in Emotion Occurrence Start Time 513. Specifically, for example, this is a time at which an emotion measured value reaches an emotion measured value written in Emotion Measured Value 515 after changing from a different emotion measured value.

If the same emotion measured value (an emotion measured value written in Emotion Measured Value 515) has been measured consecutively, the end time of that measurement time—that is, the time in which an emotion indicated by that emotion measured value occurred—is written in Emotion Occurrence End Time 514. Specifically, for example, this is a time at which an emotion measured value changes from an emotion measured value written in Emotion Measured Value 515 to a different emotion measured value.

An emotion measured value obtained based on biological information is written in Emotion Measured Value 515.

External environment information for a period from Emotion Occurrence Start Time 513 to Emotion Occurrence End Time 514 is written in Event 516a and Location 516b. Specifically, for example, information indicating an event attended by the user or an event that occurred in the user's environment is written in Event 516a, and information relating to the user's location is written in Location 516b. External environment information may be input by the user, or may be acquired from information received from outside by means of a mobile communication network or GPS (global positioning system).

For example, the following are written as emotion information indicated by Emotion History Information No. 511 “0001”: Emotion Measurement Date 512 “2008/3/25”, Emotion Occurrence Start Time 513 “12:10:00”, Emotion Occurrence End Time 514 “12:20:00”, Emotion Measured Value 515 “(−4,−2)”, Event 516a “Concert”, and Location 516b “Outdoors”. This indicates that the user was at an outdoor concert venue from 12:10 to 12:20 on Mar. 25, 2008, and emotion measured value (−4,−2) was measured from the user—that is, an emotion of sadness occurred in the user.

Provision may be made for generation of emotion information history 510 to be performed in the following way, for example. History storage section 310 monitors an emotion measured value (emotion information) input from emotion information acquisition section 220 and external environment information, and each time there is a change of any kind, creates one record based on an emotion measured value and external environment information obtained from a time when there was a change immediately before until the present. At this time, taking into consideration a case in which the same emotion measured value and external environment information continue for a long time, an upper limit may be set for a record generation interval.

This concludes a description of emotion information accumulation stage processing. Via this emotion information accumulation stage processing, past emotion information is accumulated in content editing apparatus 100 as an emotion information history.

Next, content editing stage processing will be described.

After setting has been completed for the above-described sensor and digital video camera, operation of content editing apparatus 100 is started.

In step S1400 in FIG. 8, content recording section 410 starts recording of experience video content continuously shot by the digital video camera, and output of recorded experience video content to content editing section 420.

Then, in step S1500, reference emotion characteristic acquisition section 320 executes reference emotion characteristic acquisition processing. Reference emotion characteristic acquisition processing is processing whereby a reference emotion characteristic is calculated based on an emotion information history of a reference time.

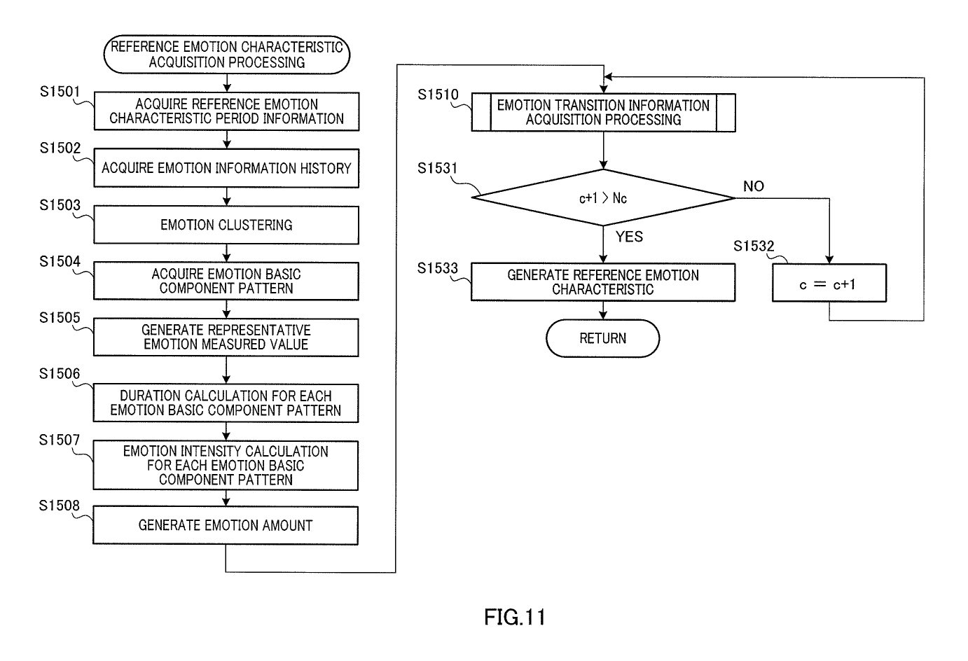

FIG. 11 is a flowchart showing reference emotion characteristic acquisition processing.

First, in step S1501, reference emotion characteristic acquisition section 320 acquires reference emotion characteristic period information. Reference emotion characteristic period information specifies a reference period.

It is desirable for a period in which a user is in a normal state, or a period of sufficient length to be able to be considered as a normal state when user states are averaged, to be set as a reference period. Specifically, a period up to a point in time going back a predetermined length of time, such as a week, six months, a year, or the like, from a point in time at which a user shoots experience video (the present) is set as a reference time. This length of time may be specified by the user, or may be a preset default value, for example.

Also, an arbitrary past period distant from the present may be set as a reference period. For example, a reference period may be the same time period as a time period in which experience video of another day was shot, or a period when the user was at the same location as an experience video shooting location in the past. Specifically, for example, this is a period in which Event 516a and Location 516b best match an event attended by the user and its location in a measurement period. A decision on a reference time can also be made based on various kinds of other information. For example, a period in which external environment information relating to a time period, such as whether an event took place in the daytime or at night, may be decided upon as a reference time.

Then, in step S1502, reference emotion characteristic acquisition section 320 acquires all emotion information corresponding to a reference emotion characteristic period within the emotion information history stored in history storage section 310. Specifically, for each point in time of a predetermined time interval, reference emotion characteristic acquisition section 320 acquires a record of the corresponding point in time from the emotion information history.

Then, in step S1503, reference emotion characteristic acquisition section 320 performs clustering relating to emotion type for an acquired plurality of records. Clustering is performed by classifying records into the emotion types shown in FIG. 2 or types conforming to these (hereinafter referred to as “classes”). By this means, an emotion measured value of a record during a reference period can be reflected in an emotion model space in a state in which a time component has been eliminated.

Then, in step S1504, reference emotion characteristic acquisition section 320 acquires an emotion basic component pattern from the results of clustering. Here, an emotion basic component pattern is a collection of a plurality of cluster members (here, records) calculated on a cluster-by-cluster basis, comprising information indicating which record corresponds to which cluster. If a variable for identifying a cluster is designated c (with an initial value of 1), a cluster is designated p_{c}, and the number of clusters is designated N_{c}, emotion basic component pattern P is expressed by equation 7 below.

[7]

P={p₁, p₂, . . . p_{c}, . . . , p_{N}_{_{c}}} (Equation 7)

If cluster p_{c} comprises cluster member representative point coordinates (that is, emotion measured value) (x_{c}, y_{c}) and cluster member emotion information history number Num, and the corresponding number of records (that is, the number of cluster members) is designated m, p_{c} is expressed by equation 8 below.

[8]

p_{c}={x_{c}, y_{c}, {Num₁, Num₂, . . . , Numₘ}} (Equation 8)

Provision may also be made for reference emotion characteristic acquisition section 320 not to use a cluster for which corresponding number of records m is less than a threshold value as an emotion basic component pattern P cluster. By this means, for example, the subsequent processing load can be reduced, and only an emotion type that passes through in the process of emotion transition can be excluded from the objects of processing.

Then, in step S1505, reference emotion characteristic acquisition section 320 calculates a representative emotion measured value. A representative emotion measured value is an emotion measured value that represents emotion measured values of a reference period, being, for example, coordinates (x_{c}, y_{c}) of a cluster for which the number of cluster members is greatest, or a cluster for which duration described later herein is longest.

Then, in step S1506, reference emotion characteristic acquisition section 320 calculates duration T for each cluster of acquired emotion basic component pattern P. Duration T is an aggregate of average values t_{c} of emotion measured value duration (that is, the difference between an emotion occurrence start time and emotion occurrence end time) calculated on a cluster-by-cluster basis, and is expressed by equation 9 below.

[9]

T={t₁, t₂, . . . , t_{c}, . . . , t_{N}_{_{c}}} (Equation 9)

If the duration of a cluster member is designated t_{cm}, average value t_{c} of the duration of cluster p_{c} is calculated, for example, by means of equation 10 below.

[10]

$${t}_{c} = \frac{{\sum }_{m = 1}^{{N}_{m}} {t}_{cm}}{{N}_{m}} \qquad ( Equation 10 )$$

For duration average value tⱼ, provision may also be made for a representative point to be decided upon from among cluster members, and for the duration of an emotion corresponding to the decided representative point to be used.

Then, in step S1507, reference emotion characteristic acquisition section 320 calculates emotion intensity H for each cluster of emotion basic component pattern P. Emotion intensity H is an aggregate of average values h_{c} obtained by averaging emotion intensity calculated on a cluster-by-cluster basis, and is expressed by equation 11 below.

[11]

H={h₁, h₂, . . . , h_{c}, . . . , h_{N}_{_{c}}} (Equation 11)

If the emotion intensity of a cluster member is designated y_{cm}, emotion intensity average value h_{c} is expressed by equation 12 below.

[12]

$${h}_{c} = \frac{{\sum }_{m = 1}^{{N}_{m}} {y}_{cm}}{{N}_{m}} \qquad ( Equation 12 )$$

If an emotion measured value is expressed as 3-dimensional emotion model space coordinate values (x_{cm}, y_{cm}, z_{cm}), emotion intensity may be a value calculated by means of equation 13 below, for example.

[13]

$${h}_{c} = \frac{{\sum }_{m = 1}^{{N}_{m}} ) \overline{{x}_{cm}^{2} + {y}_{cm}^{2} + {z}_{cm}^{2}}}{{N}_{m}} \qquad ( Equation 13 )$$

For emotion intensity average value h_{c}, provision may also be made for a representative point to be decided upon from among cluster members, and for emotion intensity corresponding to the decided representative point to be used.

Then, in step S1508, reference emotion characteristic acquisition section 320 performs emotion amount generation as shown in FIG. 5. Specifically, reference emotion characteristic acquisition section 320 performs time integration of emotion amounts in a reference period using calculated duration T and emotion intensity H.

Then, in step S1510, reference emotion characteristic acquisition section 320 performs emotion transition information acquisition processing. Emotion transition information acquisition processing is processing whereby emotion transition information is acquired.

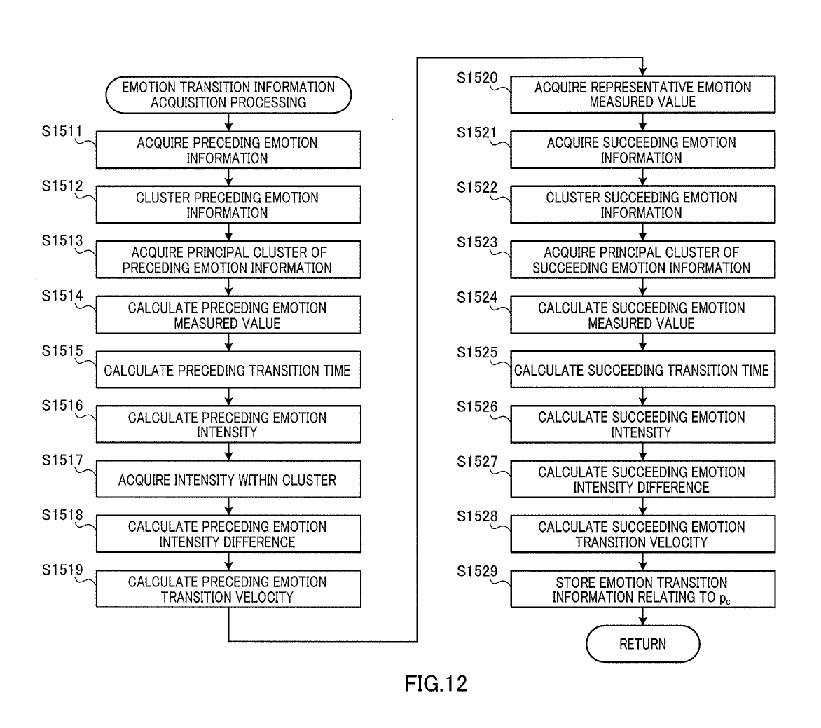

FIG. 12 is a flowchart showing emotion transition information acquisition processing.

First, in step S1511, reference emotion characteristic acquisition section 320 acquires preceding emotion information for each of the cluster members of cluster p_{c}. Preceding emotion information is pre-transition emotion information—that is, the preceding record—for the individual cluster members of cluster p_{c}. Below, information relating to cluster p_{c} under consideration is denoted by “processing-object”, and information relating to the immediately preceding record is denoted by “preceding”.

Then, in step S1512, reference emotion characteristic acquisition section 320 performs the same kind of clustering as in step S1503 in FIG. 11 on acquired preceding emotion information, and acquires a preceding emotion basic component pattern in the same way as in step S1504 in FIG. 11.

Then, in step S1513, reference emotion characteristic acquisition section 320 acquires the principal cluster of preceding emotion information. The principal cluster is, for example, a cluster for which the number of cluster members is largest, or a cluster for which duration T is longest.

Then, in step S1514, reference emotion characteristic acquisition section 320 calculates preceding emotion measured value e_{αBefore}. Preceding emotion measured value e_{αBefore} is an emotion measured value of a representative point in the principal cluster of acquired preceding emotion information.

Then, in step S1515, reference emotion characteristic acquisition section 320 calculates a preceding transition time. A preceding transition time is an average value of cluster member transition times.

Then, in step S1516, reference emotion characteristic acquisition section 320 calculates preceding emotion intensity. Preceding emotion intensity is emotion intensity for acquired preceding emotion information, and is calculated by means of the same kind of method as in step S1507 in FIG. 11.

Then, in step S1517, reference emotion characteristic acquisition section 320 acquires emotion intensity within a cluster by means of the same kind of method as in step S1507 in FIG. 11, or from the calculation result of step S1507 in FIG. 11.

Then, in step S1518, reference emotion characteristic acquisition section 320 calculates a preceding emotion intensity difference. A preceding emotion intensity difference is the difference of a processing-object emotion intensity (the emotion intensity calculated in step S1507 in FIG. 11) with respect to the preceding emotion intensity (the emotion intensity calculated in step S1516). If a preceding emotion intensity is designated H_{Before} and preceding emotion intensity is designated H, emotion intensity difference ΔH is calculated by means of equation 14 below.

[14]

ΔH=|H−H_{Before}| (Equation 14)

Then, in step S1519, reference emotion characteristic acquisition section 320 calculates a preceding emotion transition velocity. A preceding emotion transition velocity is a change in emotion intensity per unit time when making a transition from a preceding emotion type to a processing-object emotion type. If a transition time is designated ΔT, preceding emotion transition velocity e_{velBefore} is calculated by means of equation 15 below.

[15]

e_{velBefore}=ΔH/ΔT (Equation 15)

Then, in step S1520, reference emotion characteristic acquisition section 320 acquires a representative emotion measured value of processing-object emotion information by means of the same kind of method as in step S1505 in FIG. 11, or from the calculation result of step S1505 in FIG. 11.

Here, succeeding emotion information means emotion information after a transition of a cluster member of cluster p_{c}—that is, the record immediately succeeding a record for a cluster member of cluster p_{c}, and information relating to an immediately succeeding record is denoted by “succeeding”.

In steps S1521 through S1528, reference emotion characteristic acquisition section 320 uses similar processing to that in steps S1511 through S1519 to acquire succeeding emotion information, a succeeding emotion information principal cluster, a succeeding emotion measured value, a succeeding transition time, succeeding emotion intensity, a succeeding emotion intensity difference, and succeeding emotion transition velocity. This is possible by executing the processing in steps S1511 through S1519 with processing-object emotion information replaced by preceding emotion information, and succeeding emotion information newly replaced by processing-object emotion information.

Then, in step S1529, reference emotion characteristic acquisition section 320 internally stores emotion transition information relating to the p_{c} cluster, and returns to the processing in FIG. 11.

In step S1531 in FIG. 11, reference emotion characteristic acquisition section 320 determines whether or not a value resulting from adding 1 to variable c exceeds number of clusters N_{c}, and if the above value does not exceed number N_{c} (step S1531: NO), proceeds to step S1532.

In step S1532, reference emotion characteristic acquisition section 320 increments variable c by 1, returns to step S1510, and executes emotion transition information acquisition processing with the next cluster as a processing object.

On the other hand, if a value resulting from adding 1 to variable c exceeds number of clusters N_{c}—that is, if emotion transition information acquisition processing is completed for all emotion information of the reference period—(step S1531: YES), reference emotion characteristic acquisition section 320 proceeds to step S1533.

In step S1533, reference emotion characteristic acquisition section 320 generates a reference emotion characteristic based on information acquired by emotion transition information acquisition processing, and returns to the processing in FIG. 8. A set of reference emotion characteristics is generated equivalent to the number of clusters.

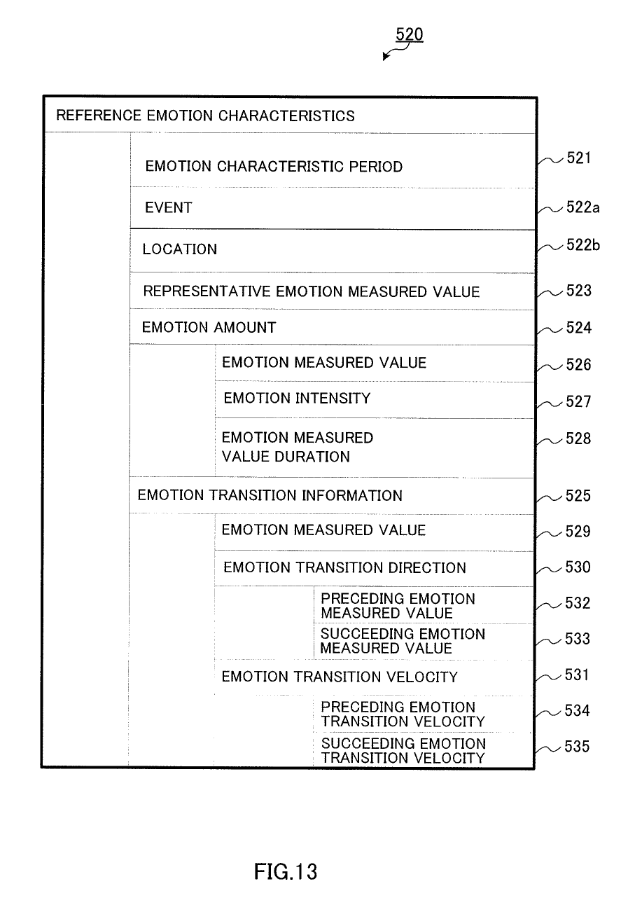

FIG. 13 is a drawing showing an example of reference emotion characteristic contents.

As shown in FIG. 13, reference emotion characteristics 520 include Emotion Characteristic Period 521, Event 522a, Location 522b, Representative Emotion Measured Value 523, Emotion Amount 524, and Emotion Transition Information 525. Emotion Amount 524 includes Emotion Measured Value 526, Emotion Intensity 527, and Emotion Measured Value Duration 528. Emotion Transition Information 525 includes Emotion Measured Value 529, Emotion Transition Direction 530, and Emotion Transition Velocity 531. Emotion Transition Direction 530 comprises a pair of items, Preceding Emotion Measured Value 532 and Succeeding Emotion Measured Value 533. Emotion Transition Velocity 531 comprises a pair of items, Preceding Emotion Transition Velocity 534 and Succeeding Emotion Transition Velocity 535.

A representative emotion measured value is used when finding emotion measured value difference r_{α} explained in FIG. 3. An emotion amount is used when finding emotion amount difference r_{β} explained in FIG. 5. Emotion transition information is used when finding emotion transition information difference r_{δ} explained in FIG. 6 and FIG. 7.

In step S1600 in FIG. 8, reference emotion characteristic acquisition section 320 records a calculated reference emotion characteristic.

If the reference time is fixed, provision may be made for the processing in steps S1100 through S1600 to be executed beforehand, and for generated reference emotion characteristics to be accumulated in reference emotion characteristic acquisition section 320 or impression degree calculation section 340.

Then, in step S1700, biological information measurement section 210 measures a user's biological information when shooting experience video, and outputs acquired biological information to emotion information acquisition section 220, in the same way as in step S1100.

Then, in step S1800, emotion information acquisition section 220 starts the emotion information acquisition processing shown in FIG. 9, in the same way as in step S1200. Emotion information acquisition section 220 may also execute emotion information acquisition processing consecutively by passing through steps S1200 and S1800.

Then, in step S1900, emotion information storage section 330 stores emotion information up to a point in time going back a predetermined unit time from the present among emotion information input every n seconds as emotion information data.

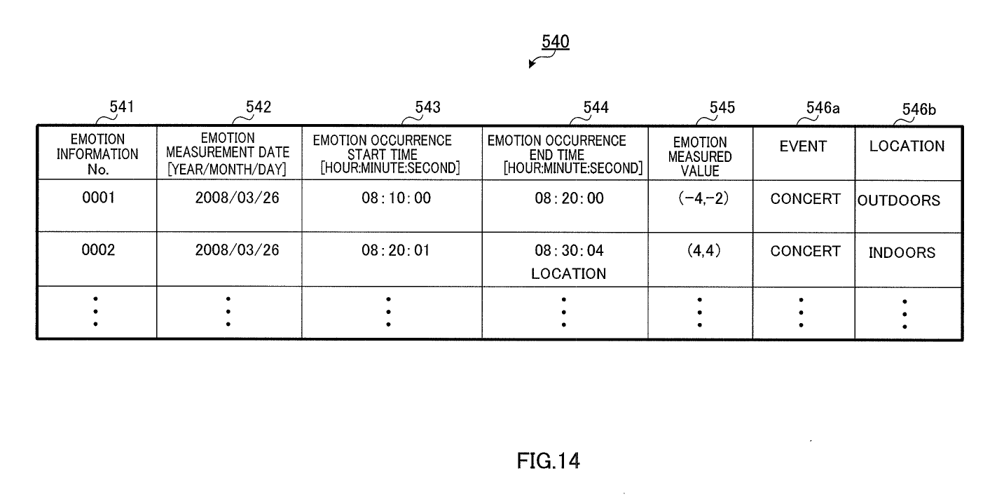

FIG. 14 is a drawing showing an example of emotion information data contents stored in step S1900 in FIG. 8.

As shown in FIG. 14, emotion information storage section 330 generates emotion information data 540 comprising records in which other information has been added to input emotion information. Emotion information data 540 has a similar configuration to emotion information history 510 shown in FIG. 10. Emotion information data 540 includes Emotion Information Number 541, Emotion Measurement Date [Year/Month/Day] 542, Emotion Occurrence Start Time [Hour:Minute:Second] 543, Emotion Occurrence End Time [Hour:Minute:Second] 544, Emotion Measured Value 545, Event 546a, and Location 546b.

Emotion information data 540 generation is performed, for example, by means of n-second-interval emotion information recording and emotion merging processing, in the same way as an emotion information history. Alternatively, emotion information data 540 generation may be performed in the following way, for example. Emotion information storage section 330 monitors an emotion measured value (emotion information) input from emotion information acquisition section 220 and external environment information, and each time there is a change of any kind, creates one emotion information data 540 record based on an emotion measured value and external environment information obtained from a time when there was a change immediately before until the present. At this time, taking into consideration a case in which the same emotion measured value and external environment information continue for a long time, an upper limit may be set for a record generation interval.

The number of emotion information data 540 records is smaller than the number of emotion information history 510 records, and is kept to a number necessary to calculate the latest measured emotion characteristic. Specifically, emotion information storage section 330 deletes the oldest record when adding a new record, and updates Emotion Information Number 541 of each record, to prevent the number of records from exceeding a predetermined upper limit on the number of records. By this means, an increase in the data size can be prevented, and processing can be performed based on Emotion Information Number 541.

In step S2000 in FIG. 8, impression degree calculation section 340 starts impression degree calculation processing. Impression degree calculation processing is processing whereby an impression degree is output based on reference emotion characteristics 520 and emotion information data 540.

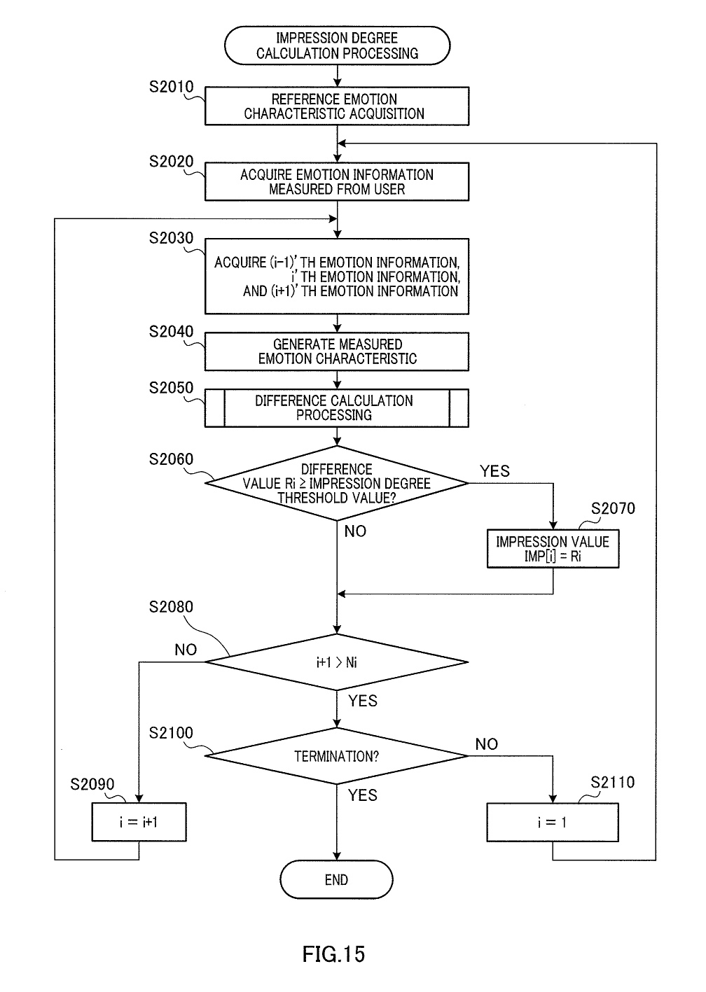

FIG. 15 is a flowchart showing impression degree calculation processing.

First, in step S2010, impression degree calculation section 340 acquires a reference emotion characteristic.

Then, in step S2020, impression degree calculation section 340 acquires emotion information data 540 measured from the user from emotion information storage section 330.

Then, in step S2030, impression degree calculation section 340 acquires (i−1)'th emotion information, i'th emotion information, and (i+1)'th emotion information, in emotion information data 540. If (i−1)'th emotion information or (i+1)'th emotion information does not exist, impression degree calculation section 340 sets a value representing an acquisition result to NULL.

Then, in step S2040, impression degree calculation section 340 generates a measured emotion characteristic in measured emotion characteristic acquisition section 341. A measured emotion characteristic comprises the same kind of items of information as a reference emotion characteristic shown in FIG. 13. Measured emotion characteristic acquisition section 341 calculates a measured emotion characteristic by executing the same kind of processing as in FIG. 12 with a processing object replaced by emotion information data.

Then, in step S2050, impression degree calculation section 340 executes difference calculation processing. The difference calculation processing refers to processing of calculating the difference of measured emotion characteristics with respect to reference emotion characteristics.

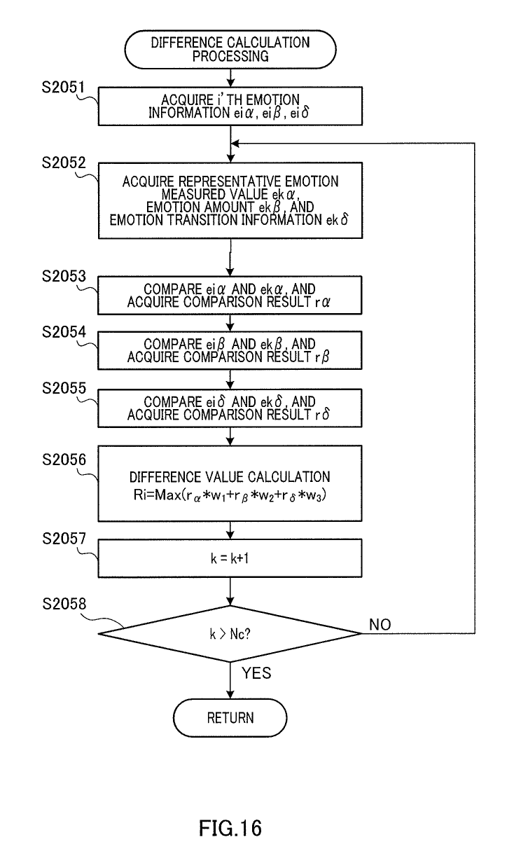

FIG. 16 is a flowchart showing an example of difference calculation processing.

First, in step S2051, impression degree calculation section 340 acquires representative emotion measured value e_{iα} emotion amount e_{iβ}, and emotion transition information e_{iδ}, from reference emotion characteristics calculated for i'th emotion information.

Then, in step S2052, impression degree calculation section 340 acquires representative emotion measured value e_{kα}, emotion amount e_{kβ}, and emotion transition information e_{kδ}, from reference emotion characteristics calculated for k'th emotion information, where k is a variable for identifying emotion information—that is, a variable for identifying a cluster—and has an initial value of 1.

Then, in step S2053, impression degree calculation section 340 compares measured emotion characteristic i'th representative emotion measured value e_{iα} with reference emotion characteristic k'th representative emotion measured value e_{kα}, and acquires emotion measured value difference r_{α} explained in FIG. 5 as the result of this comparison.

Then, in step S2054, impression degree calculation section 340 compares measured emotion characteristic i'th emotion amount e_{iβ} with reference emotion characteristic k'th emotion amount e_{kβ}, and acquires emotion amount difference r_{β} explained in FIG. 3 as the result of this comparison.

Then, in step S2055, impression degree calculation section 340 compares emotion characteristic i'th emotion transition information e_{iδ} with reference emotion characteristic k'th emotion transition information e_{kδ}, and acquires emotion transition information difference r_{δ} explained in FIG. 6 and FIG. 7 as the result of this comparison.

Then, in step S2056, impression degree calculation section 340 calculates a difference value. A difference value is a value that denotes a degree of difference of emotion information by integrating emotion measured value difference r_{α}, emotion amount difference r_{β}, and emotion transition information difference r_{δ}. Specifically, for example, a difference value is the maximum value of the sum of individually weighted emotion measured value difference r_{α}, emotion amount difference r_{β}, and emotion transition information difference r_{δ}. If the weights of emotion measured value difference r_{α}, emotion amount difference r_{β}, and emotion transition information difference r_{δ} are designated w₁, w₂, and w₃, respectively, difference value Rᵢ is calculated by means of equation 16 below.

[16]

Rᵢ=Max(r_{α}×w₁+r_{β}×w₂+r_{δ}×w₃) (Equation 16)

Weights w₁, w₂, and w₃ may be fixed values, or may be values that can be adjusted by the user.

Then, in step S2057, impression degree calculation section 340 increments variable k by 1.

Then, in step S2058, impression degree calculation section 340 determines whether or not variable k exceeds number of clusters N_{c}. If variable k does not exceed number of clusters N_{c} (step S2058: NO), impression degree calculation section 340 returns to step S2052, whereas if variable k exceeds number of clusters N_{c} (step S2058: YES), impression degree calculation section 340 returns to the processing in FIG. 15.

Thus, by means of difference calculation processing, the largest value among difference values when variable k is changed is finally acquired as difference value Rᵢ.

In step S2060 in FIG. 15, impression degree calculation section 340 determines whether or not acquired difference value Rᵢ is greater than or equal to a predetermined impression degree threshold value. The impression degree threshold value is the minimum value of difference value Rᵢ for which a user should be determined to have received a strong impression. The impression degree threshold value may be a fixed value, may be a value that can be adjusted by the user, or may be decided by experience or learning. If difference value Rᵢ is greater than or equal to the impression degree threshold value (step S2060: YES), impression degree calculation section 340 proceeds to step S2070, whereas if difference value Rᵢ is less than the impression degree threshold value (step S2060: NO), impression degree calculation section 340 proceeds to step S2080.

In step S2070, impression degree calculation section 340 sets difference value Rᵢ to impression value IMP[i]. Impression value IMP[i] is consequently a value that is a degree indicating the intensity of an impression received by a user at the time of measurement with respect to the intensity of an impression received by a user in a reference period. Moreover, impression value IMP[i] is a value that reflects an emotion measured value difference, emotion amount difference, and emotion transition information difference.

In step S2080, impression degree calculation section 340 determines whether or not a value resulting from adding 1 to variable i exceeds number of items of emotion information N₁—that is, whether or not processing has ended for all emotion information of the measurement period. Then, if the above value does not exceed number of items of emotion information Nᵢ (step S2080: NO), impression degree calculation section 340 proceeds to step S2090.

In step S2090, impression degree calculation section 340 increments variable i by 1, and returns to step S2030.

Step S2030 through step S2090 are repeated, and when a value resulting from adding 1 to variable i exceeds number of items of emotion information Nᵢ (step S2080: YES), impression degree calculation section 340 proceeds to step S2100.

In step S2100, impression degree calculation section 340 determines whether or not content recording section 410 operation has ended, for instance, and termination of impression degree calculation processing has been directed, and if termination has not been directed (step S2100: NO), proceeds to step S2110.

In step S2110, impression degree calculation section 340 restores variable i to its initial value of 1, and when a predetermined unit time has elapsed after executing the previous step S2020 processing, returns to step S2020.

On the other hand, if termination of impression degree calculation processing has been directed (step S2100: YES), impression degree calculation section 340 terminates the series of processing steps.

By means of this kind of impression degree calculation processing, an impression value is calculated every predetermined unit time for a section in which a user received a strong impression. Impression degree calculation section 340 generates impression degree information that provides correspondence of a measurement time of emotion information that is the basis of impression value calculation to a calculated impression value.

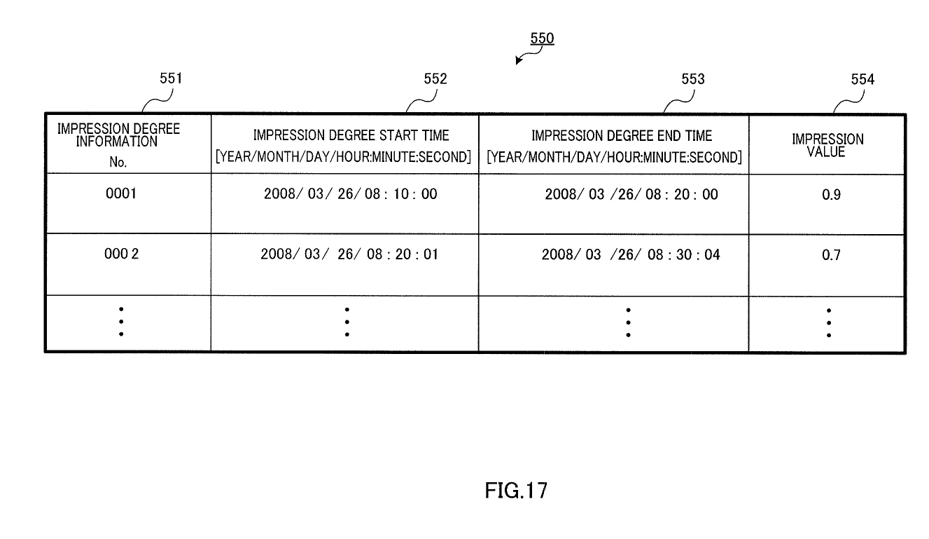

FIG. 17 is a drawing showing an example of impression degree information contents.

As shown in FIG. 17, impression degree information 550 includes Impression Degree Information Number 551, Impression Degree Start Time 552, Impression Degree End Time 553, and Impression Value 554.

If the same impression value (the impression value written in Impression Value 554) has been measured consecutively, the start time of that measurement time is written in Impression Degree Start Time.

If the same impression value (the impression value written in Impression Value 554) has been measured consecutively, the end time of that measurement time is written in Impression Degree End Time.

Impression value IMP[i] calculated by impression degree calculation processing is written in Impression Value 554.

Here, for example, Impression Value 554 “0.9” corresponding to Impression Degree Start Time 552 “2008/03/26/08:10:00” and Impression Degree End Time 553 “2008/03/26/08:20:00” is written in the record of Impression Degree Information Number 551 “0001”. This indicates that the degree of an impression received by the user from 8:10 on Mar. 26, 2008 to 8:20 on Mar. 26, 2008 corresponds to impression value “0.9”. Also, Impression Value 554 “0.7” corresponding to Impression Degree Start Time 552 “2008/03/26/08:20:01” and Impression Degree End Time 553 “2008/03/26/08:30:04” is written in the record of Impression Degree Information Number 551 “0002”. This indicates that the degree of an impression received by the user from 8:20:01 on Mar. 26, 2008 to 8:30:04 on Mar. 26, 2008 corresponds to impression value “0.7”. An impression value is larger the greater the difference between a reference emotion characteristic and a measured emotion characteristic. Therefore, this impression degree information 550 indicates that the user received a stronger impression in a section corresponding to Impression Degree Information Number 551 “0001” than in a section corresponding to Impression Degree Information Number 551 “0002”.

By referencing this kind of impression degree information, it is possible to determine immediately the degree of an impression received by the user for each point in time. Impression degree calculation section 340 stores generated impression degree information in a state in which it can be referenced by content editing section 420. Alternatively, impression degree calculation section 340 outputs an impression degree information 550 record to content editing section 420 each time a record is created, or outputs impression degree information 550 to content editing section 420 after content recording ends.

By means of the above processing, experience video content recorded by content recording section 410 and impression degree information generated by impression degree calculation section 340 are input to content editing section 420.

In step S2200 in FIG. 8, content editing section 420 executes experience video editing processing. Experience video editing processing is processing whereby a scene corresponding to a high-impression-degree period—that is, a period in which Impression Value 554 is higher than a predetermined threshold value—is extracted from experience video content, and an experience video content summary video is generated.

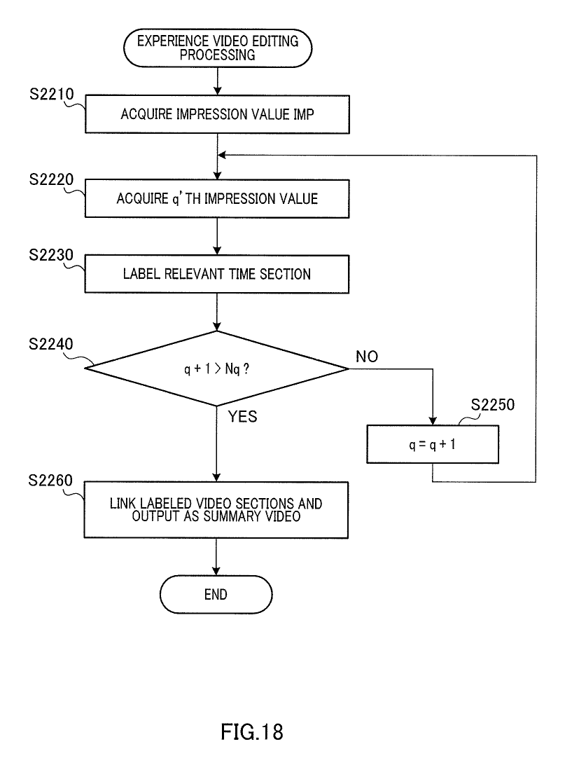

FIG. 18 is a flowchart showing an example of experience video editing processing.

First, in step S2210 content editing section 420 acquires impression degree information. Below, a variable for identifying an impression degree information record is designated q, and the number of impression degree information records is designated N_{q}. Variable q has an initial value of 1.

Then, in step S2220, content editing section 420 acquires an impression value of the q'th record.

Then, in step S2230, content editing section 420 performs labeling of a scene of a section corresponding to a period of the q'th record among experience video content using an acquired impression value. Specifically, for example, content editing section 420 adds an impression degree level to each scene as information indicating the importance of that scene.

Then, in step S2240, content editing section 420 determines whether or not a value resulting from adding 1 to variable q exceeds number of records N_{q}, and proceeds to step S2250 if that value does not exceed number of records N_{q} (step S2240: NO), or proceeds to step S2260 if that value exceeds number of records N_{q} (step S2240: YES).

In step S2250, content editing section 420 increments variable q by 1, and returns to step S2220.

On the other hand, in step S2260, content editing section 420 divides video sections of labeled experience video content, and links together divided video sections based on their labels. Then content editing section 420, outputs linked video to a recording medium, for example, as a summary video, and terminates the series of processing steps. Specifically, for example, content editing section 420 picks up only video sections to which a label indicating high scene importance is attached, and links together the picked-up video sections in time order according to the basic experience video content.

In this way, content editing apparatus 100 can select scenes for which a user received a strong impression from within experience video content with a high degree of precision, and can generate a summary video from the selected scenes.

As described above, according to this embodiment, an impression degree is calculated by means of a comparison of characteristic values based on biological information, and therefore an impression degree can be extracted without particularly imposing a burden on a user. Also, an impression degree is calculated taking a reference emotion characteristic obtained from biological information of a user himself in a reference period as a reference, enabling an impression degree to be calculated with a high degree of precision. Furthermore, a summary video is generated by selecting a scene from experience video content based on an impression degree, enabling experience video content to be edited by picking up only a scene with which a user is satisfied. Moreover, since an impression degree is extracted with a high degree of precision, content editing results with which a user is more satisfied can be obtained, and the necessity of a user performing re-editing can be reduced.

Also, a difference in emotion between a reference period and a measurement period is determined, taking into consideration differences in emotion measured values, emotion amounts, and emotion transition information subject to comparison, enabling an impression degree to be determined with a high degree of precision.

A content acquisition location and use of an extracted impression degree are not limited to those described above. For example, provision may also be made for a biological information sensor to be attached to a hotel guest, restaurant customer, or the like, and for conditions when an impression degree changes to be recorded while the experience of that person when receiving service is being shot with a camera. In this case, the quality of service can easily be analyzed by the hotel or restaurant management based on the recorded results.

### Embodiment 2

As Embodiment 2, a case will be described in which the present invention is applied to game content that performs selective operation of a portable game terminal. An impression degree extraction apparatus of this embodiment is provided in a portable game terminal.

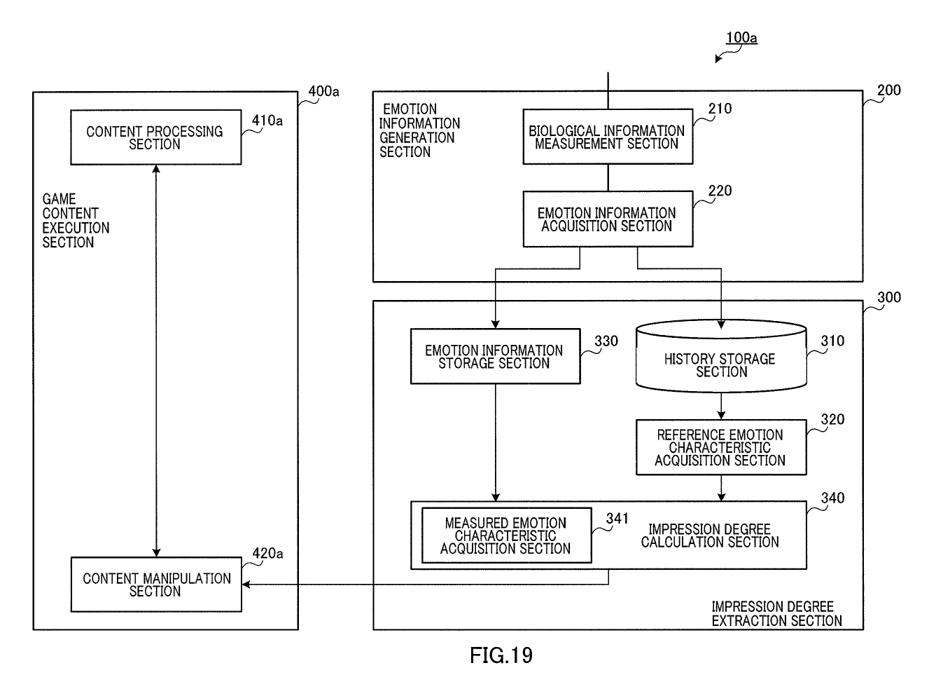

FIG. 19 is a block diagram of a game terminal that includes an impression degree extraction apparatus according to Embodiment 2 of the present invention, and corresponds to FIG. 1 of Embodiment 1. Parts identical to those in FIG. 1 are assigned the same reference codes as in FIG. 1, and duplicate descriptions thereof are omitted here.

In FIG. 19, game terminal 100a has game content execution section 400a instead of experience video content acquisition section 400 in FIG. 1.

Content execution section 400a executes game content that performs selective operation. Here, game content is assumed to be a game in which a user virtually keeps a pet, and the pet's reactions and growth differ according to manipulation contents. Game content execution section 400a has content processing section 410a and game content manipulation section 420a.

Content processing section 410a performs various kinds of processing for executing game content.

Content manipulation section 420a performs selection manipulation on content processing section 410a based on an impression degree extracted by impression degree extraction section 300. Specifically, manipulation contents for game content assigned correspondence to an impression value are set in content manipulation section 420a beforehand. Then, when game content is started by content processing section 410a and impression value calculation is started by impression degree extraction section 300, content manipulation section 420a starts content manipulation processing that automatically performs manipulation of content according to the degree of an impression received by the user.

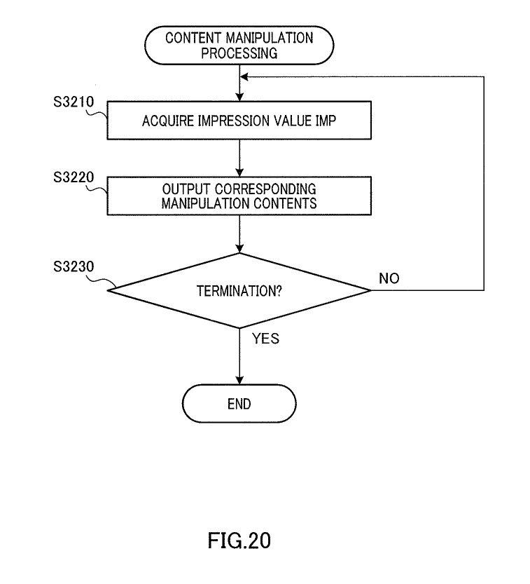

FIG. 20 is a flowchart showing an example of content manipulation processing.

First, in step S3210, content manipulation section 420a acquires impression value IMP[i] from impression degree extraction section 300. Unlike Embodiment 1, it is sufficient for content manipulation section 420a to acquire only an impression value obtained from the latest biological information from impression degree extraction section 300.

Then, in step S3220, content manipulation section 420a outputs manipulation contents corresponding to an acquired impression value to content processing section 410a.

Then, in step S3230, content manipulation section 420a determines whether processing termination has been directed, and returns to step S3210 if processing termination has not been directed (step S3230: NO), or terminates the series of processing steps if processing termination has been directed (step S3230: YES).

Thus, according to this embodiment, selection manipulation is performed on game content in accordance with the degree of an impression received by a user, without manipulation being performed manually by the user. For example, it is possible to perform unique content manipulation that differs for each user, such as content manipulation whereby, in the case of a user who normally laughs a lot, even if the user laughs an impression value does not become all that high and the pet's growth is normal, whereas in the case of a user who seldom laughs, if the user laughs an impression value becomes high and the pet's growth is rapid.

### Embodiment 3

As Embodiment 3, a case will be described in which the present invention is applied to editing of a standby screen of a mobile phone. An impression degree extraction apparatus of this embodiment is provided in a mobile phone.

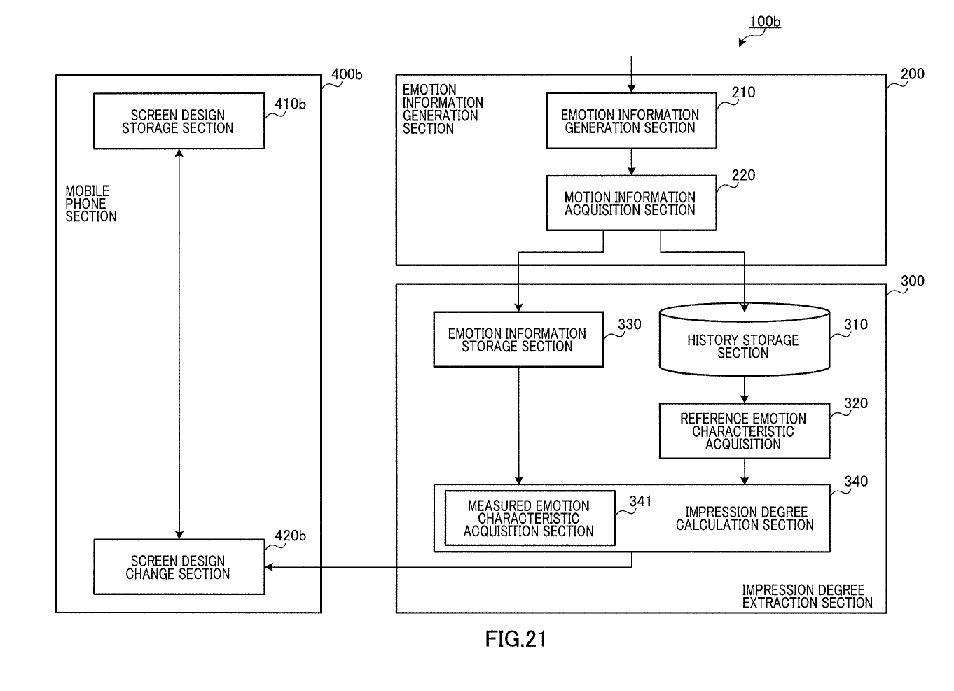

FIG. 21 is a block diagram of a mobile phone that includes an impression degree extraction apparatus according to Embodiment 3 of the present invention, and corresponds to FIG. 1 of Embodiment 1. Parts identical to those in FIG. 1 are assigned the same reference codes as in FIG. 1, and duplicate descriptions thereof are omitted here.

In FIG. 21, mobile phone 100b has mobile phone section 400b instead of experience video content acquisition section 400 in FIG. 1.

Mobile phone section 400b implements functions of a mobile phone including display control of a standby screen of a liquid crystal display (not shown). Mobile phone section 400b has screen design storage section 410b and screen design change section 420b.

Screen design storage section 410b stores a plurality of screen design data for a standby screen.

Screen design change section 420b changes the screen design of a standby screen based on an impression degree acquired by impression degree extraction section 300. Specifically, screen design change section 420b establishes correspondence between screen designs stored in screen design storage section 410b and impression values beforehand. Then screen design change section 420b executes screen design change processing whereby a screen design corresponding to the latest impression value is selected from screen design storage section 410b and applied to the standby screen.

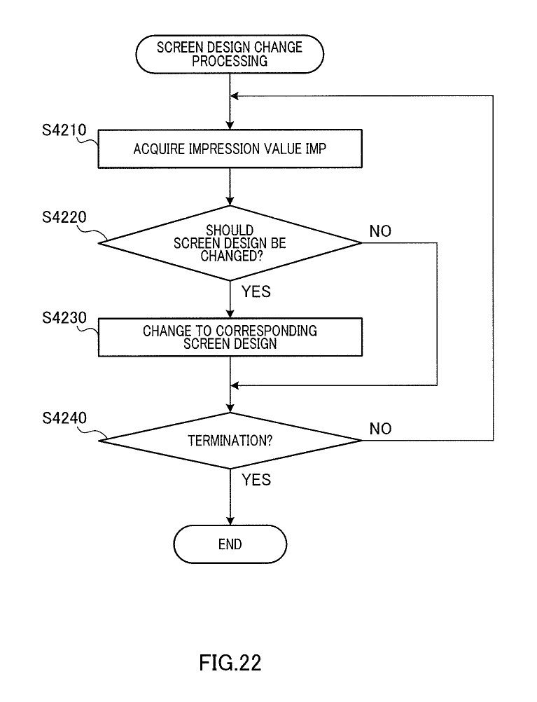

FIG. 22 is a flowchart showing an example of screen design change processing.

First, in step S4210, screen design change section 420b acquires impression value IMP[i] from impression degree extraction section 300. Unlike Embodiment 1, it is sufficient for screen design change section 420b to acquire only an impression value obtained from the latest biological information from impression degree extraction section 300. Acquisition of the latest impression value may be performed at arbitrary intervals, or may be performed each time an impression value changes.

Then, in step S4220, screen design change section 420b determines whether or not the screen design should be changed—that is, whether or not the screen design corresponding to the acquired impression value is different from the screen design currently set for the standby screen. Screen design change section 420b proceeds to step S4230 if it determines that the screen design should be changed (step S4220: YES), or proceeds to step S4240 if it determines that the screen design should not be changed (step S4220: NO).

In step S4230, screen design change section 420b acquires a standby screen design corresponding to the latest impression value from screen design storage section 410b, and changes to the screen design corresponding to the latest impression value. Specifically, screen design change section 420b acquires data of a screen design assigned correspondence to the latest impression value from screen design storage section 410b, and performs liquid crystal display screen drawing based on the acquired data.

Then, in step S4240, screen design change section 420b determines whether or not processing termination has been directed, and returns to step S4210 if termination has not been directed (step S4240: NO), or terminates the series of processing steps if termination has been directed (step S4240: YES).

Thus, according to this embodiment, a standby screen of a mobile phone can be switched to a screen design in accordance with the degree of an impression received by a user, without manipulation being performed manually by the user. Provision may also be made for screen design other than standby screen design, or an emitted color of a light emitting section using an LED (light emitting diode) or the like, to be changed according to an impression degree.

### Embodiment 4

As Embodiment 4, a case will be described in which the present invention is applied to an accessory whose design is variable. An impression degree extraction apparatus of this embodiment is provided in a communication system comprising an accessory such as a pendant head and a portable terminal that transmits an impression value to this accessory by means of radio communication.

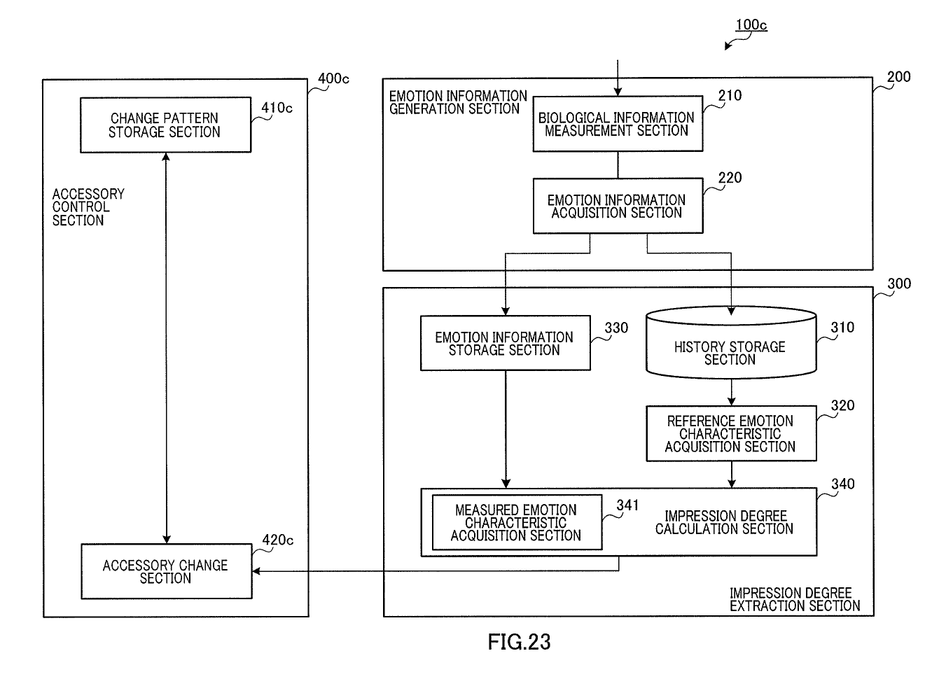

FIG. 23 is a block diagram of a communication system that includes an impression degree extraction apparatus according to Embodiment 4 of the present invention. Parts identical to those in FIG. 1 are assigned the same reference codes as in FIG. 1, and duplicate descriptions thereof are omitted here.

In FIG. 23, communication system 100c has accessory control section 400c instead of experience video content acquisition section 400 in FIG. 1.

Accessory control section 400c is incorporated into an accessory (not shown), acquires an impression degree by means of radio communication from impression degree extraction section 300 provided in a separate portable terminal, and controls the appearance of the accessory based on an acquired impression degree. The accessory has, for example, a plurality of LEDs, and is capable of changing an illuminated color or illumination pattern, or changing the design. Accessory control section 400c has change pattern storage section 410c and accessory change section 420c.

Change pattern storage section 410c stores a plurality of accessory appearance change patterns.

Accessory change section 420c changes the appearance of the accessory based on an impression degree extracted by impression degree extraction section 300. Specifically, accessory change section 420c establishes correspondence between screen designs stored in change pattern storage section 410c and impression values beforehand. Then accessory change section 420c executes accessory change processing whereby a change pattern corresponding to the latest impression value is selected from change pattern storage section 410c, and the appearance of the accessory is changed in accordance with the selected change pattern.

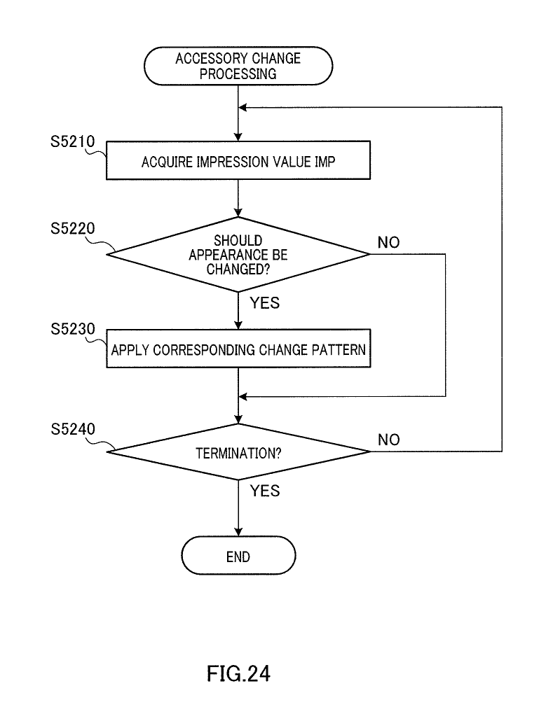

FIG. 24 is a flowchart showing an example of accessory change processing.

First, in step S5210, accessory change section 420c acquires impression value IMP[i] from impression degree extraction section 300. Unlike Embodiment 1, it is sufficient for accessory change section 420c to acquire only an impression value obtained from the latest biological information from impression degree extraction section 300. Acquisition of the latest impression value may be performed at arbitrary intervals, or may be performed each time an impression value changes.

Then, in step S5220, accessory change section 420c determines whether or not the appearance of the accessory should be changed—that is, whether or not the change pattern corresponding to the acquired impression value is different from the change pattern currently being applied. Accessory change section 420c proceeds to step S5230 if it determines that the appearance of the accessory should be changed (step S5220: YES), or proceeds to step S5240 if it determines that the appearance of the accessory should not be changed (step S5220: NO).

In step S5230, accessory change section 420c acquires a change pattern corresponding to the latest impression value from impression degree extraction section 300, and applies the change pattern corresponding to the latest impression value to the appearance of the accessory.

Then, in step S5240, accessory change section 420c determines whether or not processing termination has been directed, and returns to step S5210 if termination has not been directed (step S5240: NO), or terminates the series of processing steps if termination has been directed (step S5240: YES).

Thus, according to this embodiment, the appearance of an accessory can be changed in accordance with the degree of an impression received by a user, without manipulation being performed manually by the user. Also, the appearance of an accessory can be changed by reflecting a user's feelings by combining another emotion characteristic, such as emotion type or the like, with an impression degree. Moreover, the present invention can also be applied to an accessory other than a pendant head, such as a ring, necklace, wristwatch, and so forth. Furthermore, the present invention can also be applied to various kinds of portable goods, such as mobile phones, bags, and the like.

### Embodiment 5

As Embodiment 5, a case will be described in which content is edited using a measured emotion characteristic as well as an impression degree.

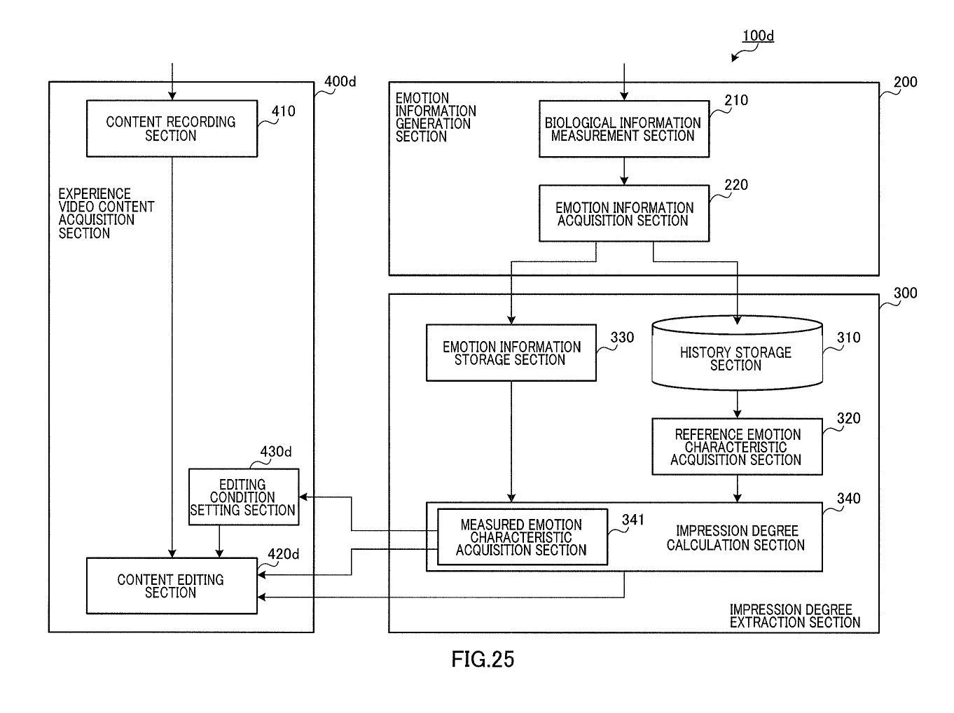

FIG. 25 is a block diagram of a content editing apparatus that includes an impression degree extraction apparatus according to Embodiment 5 of the present invention, and corresponds to FIG. 1 of Embodiment 1. Parts identical to those in FIG. 1 are assigned the same reference codes as in FIG. 1, and duplicate descriptions thereof are omitted here.

In FIG. 25, experience video content acquisition section 400d has content editing section 420d that executes different experience video editing processing from content editing section 420 in FIG. 1, and also has editing condition setting section 430d.

Editing condition setting section 430d acquires a measured emotion characteristic from measured emotion characteristic acquisition section 341, and receives an editing condition setting associated with the measured emotion characteristic from a user. An editing condition is a condition for a period for which the user desires editing. Editing condition setting section 430d performs reception of this editing condition setting using a user input screen that is a graphical user interface.

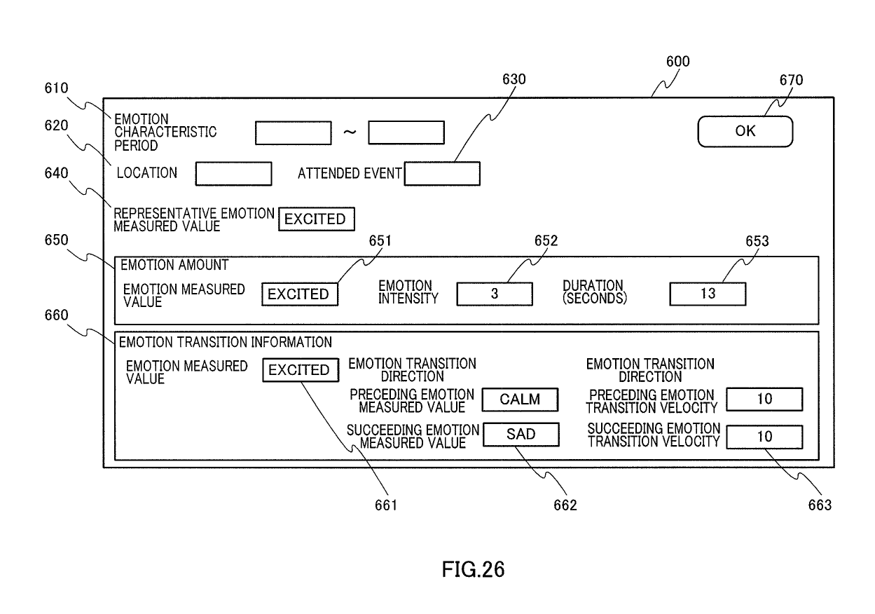

FIG. 26 is a drawing showing an example of a user input screen.

As shown in FIG. 26, user input screen 600 has period specification boxes 610, location specification box 620, attended event specification box 630, representative emotion measured value specification box 640, emotion amount specification box 650, emotion transition information specification box 660, and “OK” button 670. Boxes 610 through 660 have a pull-down menu or text input box, and receive item selection or text input by means of user manipulation of an input apparatus (not shown) such as a keyboard or mouse. That is to say, items that can be set by means of user input screen 600 correspond to measured emotion characteristic items.

Period specification boxes 610 receive a specification of a period that is an editing object from within a measurement period. Location specification box 620 receives input specifying an attribute of a location that is an editing object by means of text input. Attended event specification box 630 receives input specifying an attribute of an event that is an editing object from among attended event attributes by means of text input. Representative emotion measured value specification box 640 receives a specification of an emotion type that is an editing object by means of a pull-down menu of emotion types corresponding to representative emotion measured values.

Emotion amount specification box 650 comprises emotion measured value specification box 651, emotion intensity specification box 652, and duration specification box 653. Emotion measured value specification box 651 can also be configured linked to representative emotion measured value specification box 640. Emotion intensity specification box 652 receives input specifying a minimum value of emotion intensity that is an editing object. Duration specification box 653 receives input specifying a minimum value of duration that is an editing object for a time for which a state in which emotion intensity exceeds a specified minimum value continues by means of a pull-down menu of numeric values.

Emotion transition information specification box 660 comprises emotion measured value specification box 661, emotion transition direction specification boxes 662, and emotion transition velocity specification boxes 663. Emotion measured value specification box 661 can also be configured linked to representative emotion measured value specification box 640. Emotion transition direction specification boxes 662 receive a preceding emotion measured value and succeeding emotion measured value specification as a specification of an emotion transition direction that is an editing object by means of a pull-down menu of emotion types. Emotion transition velocity specification boxes 663 receive a preceding emotion transition velocity and succeeding emotion transition velocity specification as a specification of an emotion transition velocity that is an editing object by means of a pull-down menu of numeric values.

By manipulating this kind of user input screen 600, a user can specify a condition of a place the user considers to be memorable, associated with a measured emotion characteristic. When “OK” button 670 is pressed by the user, editing condition setting section 430d outputs screen setting contents at that time to content editing section 420d as editing conditions.

Content editing section 420d not only acquires impression degree information from impression degree calculation section 340, but also acquires a measured emotion characteristic from measured emotion characteristic acquisition section 341. Then content editing section 420d performs experience video editing processing whereby an experience video content summary video is generated based on impression degree information, a measured emotion characteristic, and an editing condition input from editing condition setting section 430d. Specifically, content editing section 420d generates an experience video content summary video by extracting only a scene corresponding to a period matching an editing condition from within a period for which an impression value is higher than a predetermined threshold value.

Alternatively, content editing section 420d may correct an impression value input from impression degree calculation section 340 according to whether or not a period matches an editing condition, and generate an experience video content summary video by extracting only a scene of a period in which the corrected impression value is higher than a predetermined threshold value.

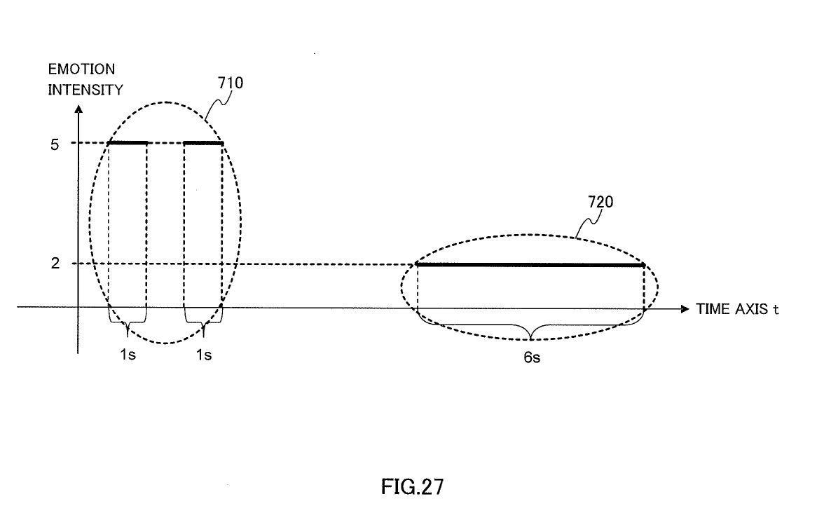

FIG. 27 is a drawing for explaining an effect obtained by limiting editing objects.

As shown in FIG. 27, in first section 710, a section in which the emotion intensity of emotion type “Excited” is 5 continues for one second, and the emotion intensity of the remainder of the section is low.

Also, this duration is short to the same degree as when emotion intensity temporarily becomes high in a normal state. In such a case, first section 710 should be excluded from editing objects. On the other hand, in second section 720, a section in which emotion intensity is 2 continues for six seconds. Although emotion intensity is low, this duration is longer than duration in a normal state. In this case, second section 720 should be an editing object.

Thus, for example, in user input screen 600 shown in FIG. 6, a user sets “Excited” in representative emotion measured value specification box 640, “3” in emotion intensity specification box 652 of emotion amount specification box 650, and “3” in duration specification box 653 of emotion amount specification box 650, and presses “OK” button 670. In this case, first section 710 does not satisfy the editing conditions and is therefore excluded from editing objects, whereas second section 720 satisfies the editing conditions and therefore becomes an editing object.

Thus, according to this embodiment, content can be automatically edited by picking up a place that a user considers to be memorable. Also, a user can specify an editing condition associated with a measured emotion characteristic, enabling a user's subjective emotion to be reflected more accurately in content editing. Moreover, the precision of impression degree extraction can be further improved if an impression value is corrected based on an editing condition.

Editing condition setting section 430d may also include a condition that is not directly related to a measured emotion characteristic in editing conditions. Specifically, for example, editing condition setting section 430d receives a specification of an upper-limit time in a summary video. Then content editing section 420d changes the duration or emotion transition velocity of an emotion type that is an editing object within the specified range, and uses a condition that is closest to the upper-limit time. In this case, if the total time of periods satisfying other conditions does not reach the upper-limit time, editing condition setting section 430d may include a scene of lower importance (with a lower impression value) in a summary video.

A procedure of performing impression value correction or content editing using a measured emotion characteristic or the like can also be applied to Embodiment 2 through Embodiment 4.

Apart from the above-described embodiments, the present invention can also be applied to performing various kinds of selection processing in electronic devices based on a user's emotion. Examples in the case of a mobile phone are selection of a type of ringtone, selection of a call acceptance/denial state, or selection of a service type in an information distribution service.

Also, for example, by applying the present invention to a recorder that stores information obtained from an in-vehicle camera and a biological information sensor attached to a driver in associated fashion, a lapse of concentration can be detected from a change in the driver's impression value. Then, in the event of a lapse of concentration, the driver can be alerted by a voice or suchlike warning, and in the event of an accident, for instance, analysis of the cause of the accident can easily be performed by extracting video shot at the time.

Also, separate emotion information generation sections may be provided for calculating a reference emotion characteristic and for calculating a measured emotion characteristic.

The disclosure of Japanese Patent Application No. 2008-174763, filed on Jul. 3, 2008, including the specification, drawings and abstract, is incorporated herein by reference in its entirety.

### INDUSTRIAL APPLICABILITY

An impression degree extraction apparatus and impression degree extraction method according to the present invention are suitable for use as an impression degree extraction apparatus and impression degree extraction method that enable an impression degree to be extracted with a high degree of precision without particularly imposing a burden on a user. By performing impression degree calculation based on a change of psychological state, an impression degree extraction apparatus and impression degree extraction method according to the present invention can perform automatic discrimination of a user's emotion that is different from normal, and can perform automatic calculation of an impression degree faithful to a user's emotion characteristic. It is possible for a result of this calculation to be utilized in various applications, such as an automatic summary of experience video, a game, a mobile device such as a mobile phone, accessory design, an automobile-related application, a customer management system, and the like.

1. An impression degree extraction apparatus comprising:
a first emotion characteristic acquisition section that acquires a first emotion characteristic indicating a characteristic of an emotion that has occurred in a user in a first period; and
an impression degree calculation section that calculates an impression degree that is a degree indicating intensity of an impression received by the user in the first period by means of a comparison of a second emotion characteristic indicating a characteristic of an emotion that has occurred in the user in a second period different from the first period with the first emotion characteristic.

2. The impression degree extraction apparatus according to claim 1, wherein the impression degree calculation section calculates the impression degree as higher the greater a difference between the first emotion characteristic and the second emotion characteristic as a reference.

3. The impression degree extraction apparatus according to claim 1, further comprising a content editing section that performs content editing based on the impression degree.

4. The impression degree extraction apparatus according to claim 1, further comprising:
a biological information measurement section that measures biological information of the user; and
a second emotion characteristic acquisition section that acquires the second emotion characteristic, wherein:
the first emotion characteristic acquisition section acquires the first emotion characteristic from the biological information; and
the second emotion characteristic acquisition section acquires the second emotion characteristic from the biological information.

5. The impression degree extraction apparatus according to claim 1, wherein the second emotion characteristic and the first emotion characteristic are at least one of an emotion measured value indicating intensity of an emotion including arousal and valence of an emotion, an emotion amount obtained by time integration of the emotion measured value, and emotion transition information including a direction or velocity of a change of the emotion measured value.

6. The impression degree extraction apparatus according to claim 1, wherein the second period is a period in which a user is in a normal state, or a period in which external environment information is obtained that is identical to external environment information obtained in the first period.

7. The impression degree extraction apparatus according to claim 4, wherein the biological information is at least one of heart rate, pulse, body temperature, facial myoelectrical signal, voice, brainwave, electrical skin resistance, skin conductance, skin temperature, electrocardiographic frequency, and facial image, of a user.

8. The impression degree extraction apparatus according to claim 3, wherein:
the content is video content recorded in the first period; and
the editing is processing whereby a summary video is generated by extracting a scene for which an impression degree is high from the video content.

9. An impression degree extraction method comprising:
a step of acquiring a first emotion characteristic indicating a characteristic of an emotion that has occurred in a user in a first period; and
a step of calculating an impression degree that is a degree indicating intensity of an impression received by the user in the first period by means of a comparison of a second emotion characteristic indicating a characteristic of an emotion that has occurred in the user in a second period different from the first period with the first emotion characteristic.
